SEQUENCE LISTING (4 sequences)

SEQ ID NO: 1 - DNA, 6572 bp, Homo sapiens
atggtcaatgagaatacgaggatgtacattccagaggaaaaccaccaaggttccaactatgggagcccacgccccgcccatgccaacatgaatgccaatgcggcagcggggctggcccctgagcacatccccaccccgggggctgccctgtcgtggcaggcggccatcgacgcagcccggcaggctaagctgatgggcagcgctggcaatgcgaccatctccacagtcagctccacgcagcggaagcggcagcaatatgggaaacccaagaagcagggcagcaccacggccacacgcccgccccgagccctgctctgcctgaccctgaagaaccccatccggagggcctgcatcagcattgtcgaatggaaaccatttgaaataattattttactgactatttttgccaattgtgtggccttagcgatctatattccctttccagaagatgattccaacgccaccaattccaacctggaacgagtggaatatctctttctcataatttttacggtggaagcgtttttaaaagtaatcgcctatggactcctctttcaccccaatgcctacctccgcaacggctggaacctactagattttataattgtggttgtggggctttttagtgcaattttagaacaagcaaccaaagcagatggggcaaacgctctcggagggaaaggggccggatttgatgtgaaggcgctgagggccttccgcgtgctgcgccccctgcggctggtgtccggagtcccaagtctccaggtggtcctgaattccatcatcaaggccatggtccccctgctgcacatcgccctgcttgtgctgtttgtcatcatcatctacgccatcatcggcttggagctcttcatggggaagatgcacaagacctgctacaaccaggagggcatagcagatgttccagcagaagatgacccttccccttgtgcgctggaaacgggccacgggcggcagtgccagaacggcacggtgtgcaagcccggctgggatggtcccaagcacggcatcaccaactttgacaactttgccttcgccatgctcacggtgttccagtgcatcaccatggagggctggacggacgtgctgtactgggtcaatgatgccgtaggaagggactggccctggatctattttgttacactaatcatcatagggtcattttttgtacttaacttggttctcggtgtgcttagcggagagttttccaaagagagggagaaggccaaggcccggggagatttccagaagctgcgggagaagcagcagctagaagaggatctcaaaggctacctggattggatcactcaggccgaagacatcgatcctgagaatgaggacgaaggcatggatgaggagaagccccgaaacatgagcatgcccaccagtgagaccgagtccgtcaacaccgaaaacgtggctggaggtgacatcgagggagaaaactgcggggccaggctggcccaccggatctccaagtcaaagttcagccgctactggcgccggtggaatcggttctgcagaaggaagtgccgcgccgcagtcaagtctaatgtcttctactggctggtgattttcctggtgttcctcaacacgctcaccattgcctctgagcactacaaccagcccaactggctcacagaagtccaagacacggcaaacaaggccctgctggccctgttcacggcagagatgctcctgaagatgtacagcctgggcctgcaggcctacttcgtgtccctcttcaaccgctttgactgcttcgtcgtgtgtggcggcatcctggagaccatcctggtggagaccaagatcatgtccccactgggcatctccgtgctcagatgcgtccggctgctgaggattttcaagatcacgaggtactggaactccttgagcaacctggtggcatccttgctgaactctgtgcgctccatcgcctccctgctccttctcctcttcctcttcatcatcatcttctccctcctggggatgcagctctttggaggaaagttcaactttgatgagatgcagacccggaggagcacattcgataacttcccccagtccctcctcactgtgtttcagatcctgaccggggaggactggaattcggtgatgtatgatgggatcatggcttatggcggcccctcttttccagggatgttagtctgtatttacttcatcatcctcttcatctgtggaaactatatcctactgaatgtgttcttggccattgctgtggacaacctggctgatgctgagagcctcacatctgcccaaaaggaggaggaagaggagaaggagagaaagaagctggccaggactgccagcccagagaagaaacaagagttggtggagaagccggcagtgggggaatccaaggaggagaagattgagctgaaatccatcacggctgacggagagtctccacccgccaccaagatcaacatggatgacctccagcccaatgaaaatgaggataagagcccctaccccaacccagaaactacaggagaagaggatgaggaggagccagagatgcctgtcggccctcgcccacgaccactctctgagcttcaccttaaggaaaaggcagtgcccatgccagaagccagcgcgtttttcatcttcagctctaacaacaggtttcgcctccagtgccaccgcattgtcaatgacacgatcttcaccaacctgatcctcttcttcattctgctcagcagcatttccctggctgctgaggacccggtccagcacacctccttcaggaaccatatcctaggcaatgcagactatgtcttcactagtatctttacattagaaattatccttaagatgactgcttatggggctttcttgcacaagggttctttctgccggaactacttcaacatcctggacctgctggtggtcagcgtgtccctcatctcctttggcatccagtccagtgcaatcaatgtcgtgaagatcttgcgagtcctgcgagtactcaggcccctgagggccatcaacagggccaaggggctaaagcatgtggttcagtgtgtgtttgtcgccatccggaccatcgggaacatcgtgattgtcaccaccctgctgcagttcatgtttgcctgcatcggggtccagctcttcaagggaaagctgtacacctgttcagacagttccaagcagacagaggcggaatgcaagggcaactacatcacgtacaaagacggggaggttgaccaccccatcatccaaccccgcagctgggagaacagcaagtttgactttgacaatgttctggcagccatgatggccctcttcaccgtctccaccttcgaagggtggccagagctgctgtaccgctccatcgactcccacacggaagacaagggccccatctacaactaccgtgtggagatctccatcttcttcatcatctacatcatcatcatcgccttcttcatgatgaacatcttcgtgggcttcgtcatcgtcacctttcaggagcagggggagcaggagtacaagaactgtgagctggacaagaaccagcgacagtgcgtggaatacgccctcaaggcccggcccctgcggaggtacatccccaagaaccagcaccagtacaaagtgtggtacgtggtcaactccacctacttcgagtacctgatgttcgtcctcatcctgctcaacaccatctgcctggccatgcagcactacggccagagctgcctgttcaaaatcgccatgaacatcctcaacatgctcttcactggcctcttcaccgtggagatgatcctgaagctcattgccttcaaacccaagcactatttctgtgatgcatggaatacatttgacgccttgattgttgtgggtagcattgttgatatagcaatcaccgaggtaaacccagctgaacatacccaatgctctccctctatgaacgcagaggaaaactcccgcatctccatcaccttcttccgcctgttccgggtcatgcgtctggtgaagctgctgagccgtggggagggcatccggacgctgctgtggaccttcatcaagtccttccaggccctgccctatgtggccctcctgatcgtgatgctgttcttcatctacgcggtgatcgggatgcaggtgtttgggaaaattgccctgaatgataccacagagatcaaccggaacaacaactttcagaccttcccccaggccgtgctgctcctcttcaggtgtgccaccggggaggcctggcaggacatcatgctggcctgcatgccaggcaagaagtgtgccccagagtccgagcccagcaacagcacggagggtgaaacaccctgtggtagcagctttgctgtcttctacttcatcagcttctacatgctctgtgccttcctgatcatcaacctctttgtagctgtcatcatggacaactttgactacctgacaagggactggtccatccttggtccccaccacctggatgagtttaaaagaatctgggcagagtatgaccctgaagccaagggtcgtatcaaacacctggatgtggtgaccctcctccggcggattcagccgccactaggttttgggaagctgtgccctcaccgcgtggcttgcaaacgcctggtctccatgaacatgcctctgaacagcgacgggacagtcatgttcaatgccaccctgtttgccctggtcaggacggccctgaggatcaaaacagaagagggacccagcccatcagaggcccaccaaggggctgaggatcctttccgccctgcagggaacctagaacaagccaatgaggagctgcgggcgatcatcaagaagatctggaagcggaccagcatgaagctgctggaccaggtggtgccccctgcaggtgatgatgaggtcaccgttggcaagttctacgccacgttcctgatccaggagtacttccggaagttcaagaagcgcaaagagcagggccttgtgggcaagccctcccagaggaacgcgctgtctctgcaggctggcttgcgcacactgcatgacatcgggcctgagatccgacgggccatctctggagatctcaccgctgaggaggagctggacaaggccatgaaggaggctgtgtccgctgcttctgaagatgacatcttcaggagggccggtggcctgttcggcaaccacgtcagctactaccaaagcgacggccggagcgccttcccccagaccttcaccactcagcgcccgctgcacatcaacaaggcgggcagcagccagggcgacactgagtcgccatcccacgagaagctggtggactccaccttcaccccgagcagctactcgtccaccggctccaacgccaacatcaacaacgccaacaacaccgccctgggtcgcctccctcgccccgccggctaccccagcacggtcagcactgtggagggccacgggccccccttgtcccctgccatccgggtgcaggaggtggcgtggaagctcagctccaacaggtgccactcccgggagagccaggcagccatggcgggtcaggaggagacgtctcaggatgagacctatgaagtgaagatgaaccatgacacggaggcctgcagtgagcccagcctgctctccacagagatgctctcctaccaggatgacgaaaatcggcaactgacgctcccagaggaggacaagagggacatccggcaatctccgaagaggggtttcctccgctctgcctcactaggtcgaagggcctccttccacctggaatgtctgaagcgacagaaggaccgagggggagacatctctcagaagacagtcctgcccttgcatctggttcatcatcaggcattggcagtggcaggcctgagccccctcctccagagaagccattcccctgcctcattccctaggccttttgccaccccaccagccacacctggcagccgaggctggcccccacagcccgtccccaccctgcggcttgagggggtcgagtccagtgagaaactcaacagcagcttcccatccatccactgcggctcctgggctgagaccacccccggtggcgggggcagcagcgccgcccggagagtccggcccgtctccctcatggtgcccagccaggctggggccccagggaggcagttccacggcagtgccagcagcctggtggaagcggtcttgatttcagaaggactggggcagtttgctcaagatcccaagttcatcgaggtcaccacccaggagctggccgacgcctgcgacatgaccatagaggagatggagagcgcggccgacaacatcctcagcgggggcgccccacagagccccaatggcgccctcttaccctttgtgaactgcagggacgcggggcaggaccgagccgggggcgaggaggacgcgggctgtgtgcgcgcgcggggtgcaccgagtgaggaggagctccaggacagcagggtctacgtcagcagcctgtagtgggcgctgccagatgcgggcttttttttttttatttgtttcaatgttcctaatgggttcgtttcagaagtgcctcactgttctcgtgacctggagtt

SEQ ID NO: 2 - protein, 2157 aa, Homo sapiens
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILGNADYVFTSIFTLEIILKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEEGPSPSEAHQGAEDPFRPAGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGAPSEEELQDSRVYVSSL

SEQ ID NO: 3 - DNA, 6779 bp, Danio rerio
gccttttttacaaccttttcttcattcaccttcggataaagctggaccacatatatggtaaatgaaagcaaaaacatgtacataccggaggacacccttgaaaaccatcaaggctccaactattccagtccgtcgttggctccggtccccagtctgaatgaggacgagcatgtggggggcgggggtggggtattgggtcttgcacctaaacacattcccacccctggcgccccccttagttggcaggcagccattttcgcggcacggcaggccaagctgatgggcaccaccggagcaccaatctccactgcgagctccacgcagcgcaaacgccagcattacaccaaacccaagaaacaggccagcactgcctccacacgcccaccccgagcccttctctgcctcactctcaaaaaccccatccgcagggcgtgcatcaatatcgtggaatggaaaccgtttgaaatcataattttgatgactatttttgcaaactgtgtggccttagctgtctacatccctttccctgaggacgattccaacgcaaccaactcaaatctggaacgggtggagtatgggtttctgatcatttttacggtggaagcctttctgaaagttattgcttatgggctgctgtgtcaccctaatgcatacctgcggaatggctggaacttgttggatttcatcatcgtggtcgtagggctcttcagtgcaatattagagcaggccactaaaggggatggtggtacttcaatgggaggcaaagctgcagggtttgatgtaaaagcccttcgagcctttagagtgttgagacctctaagactcgtgtctggagtacctagtttacaggtggtgctgaactccatcattaaagccatggttcccctccttcacatcgctctgctcgttctgttcgtcatcatcatttatgccatcatcggcctcgagctcttcatgggcaagatgcatagaaccggtttcttctataaagatggacacaaaggtcatatagctgaagagaagccggccccctgcgctccaagctccgcccatggaagacactgctctccgcccaacataacgcagtgcatgatgggatgggagggcccaaatgatggaatcacgaactttgataactttgcatttgccatgctgactgtgtttcagtgcatcacgatggagggctggactgatgtcctgtactggatgcaggatgccatgggttatgagcttccatgggtctattttgtcagtctggtcatctttggatcctttttcgttcttaatctggttctgggtgtgttgagcggagagttctctaaagagcgagaaaaggccaaagctcgtggtgattttcagaagctccgtgagaagcagcagctggaggaggatctaaagggctacctggactggatcacgcaagcagaggatattgaccctgagaatgacgacgagggactggatgacgacaagcctagaaatctgagtatgccggccagtgaaaatgagtcagtgaacactgataatgctccagctggagacatggaaggagagacctgctgtacccgtatggcaaataggatctccaaatccaaattcagtcgatattcacgccggtggaatcggttatgtcggcgtaagtgccgtgcagcagtgaaatcaaacgtcttctactggctggtgatttttctggtatttttgaacacacttactattgcctcagagcatcaccagcagccggagtggctcaccaatgtacaagatatagccaataaggtgctgctggctctctttacgggtgagatgctgttaaagatgtacagtctgggcctacaggcctattttgtctcccttttcaaccgatttgacagctttgtggtgtgcggtgggattctggaaaccatcctggtggagaccaaaataatgtcacccctgggcatctctgtgttgcgctgcgtacgtctgcttcgcatcttcaagatcaccagatactggaactctctgagtaatctagtggcgtctctgttgaactctgtgcgttcgatcgcatccctgttactgctgctcttcctcttcattatcatcttcagtctgctcggaatgcagctctttggtggaaagttcaactttgatgagactcgacgcagcacatttgataatttccctcagtctctcctcaccgtctttcagattttgaccggagaggactggaactctgtaatgtacgatggcatcatggcgtatggagggccgtcctttcctggcatgcttgtctgcatttacttcatcatcctcttcatctgtggaaactacatcctcctgaatgtcttcttggccattgctgtggacaacctcgcagacgcagagagtctaacatcagcgcagaaagaggaagaggaggagaaagagagaaagaagctggccagaacggccagtccagagaagcgccagaactctgagaaaccacccctagaggatgaaaagaaagaggaaaaaattgaactcaaatccatcacttcagatggagagacgccaactgccaccaagatcaacatagatgagtacacaggggaggacaacgaagagaaaaatccatatcctgtcaacgatttcccagcaagggaggatgatgacgaggaggagccagagatgccaggaggtccccctccacctccactatctgacattcagctgaaggagaaagacgcccccatgccacaagacaaaaccttctttattttcagccccagcaacaacttcagggtgttgtggcataagattgtaaaccacaacatcttcaccaatttaatactgctcttcatactgctgagcagcatttcactgccagcggaggacccagtgaagaatgactcattcaggaatcagattctaggctatgcagattatgtgtttacaggaatcttcaccatagagatcatattaaagatgacagcgtatggagcatttcttcataagggttcattttgtcggaattattttaatattttggatctggtggtggtcagtgtgtctctgatctcctctggaattcagtcaagtgccattaatgtagtgaagattctcagagtgctgagggtgttgaggcccctgagagcaatcaacagagccaagggcctcaaacatgtggttcagtgtgtgtttgttgccatccgcaccatcgggaatatcgtcatcgtcacctctttgctgcagttcatgtttgcctgtattggtgttcaactcttaaagggcaaattcttctactgcacagacacttctaaacagacgcaggctgagtgcagaggggcctatatactgtacaaggatgggaatgttggagagccagagaaagctcaacgctcctgggagaacagtgacttcaactttgatgatgtcctacagggcatgatggctctgtttgctgtgtccacttttgagggttggccagggctcctctacagagccatcgactcccatgcagaggatgttgggccgatctacaactaccgtgtggtcatctccatattcttcatcatctacattatcatcatcgcattcttcatgatgaacatattcgtaggtttcgtcattgtgacgtttcaggagcagggagagcaagagtacaaaaactgtgagctggacaagaaccagcgtcagtgtgtggagtatgccctgaaagctcgtcccctacgcaggtacattcccaaaaacccatatcagtacaaagtctggtacgtggtgaactctacctactttgagtacctgatgttcacgctcatccttctcaacaccatctgcctggccatgcagcaccatggtcaatctcagtctttcaacaaagccatgaatatcctcaatatgttgttcactggcctcttcactgtggaaatgatcctcaaactcatcgctttcaaacccaggcattattttgttgatgcatggaacacgtttgatgcccttattgtagtgggtagtgttgttgatatagccatcacagaggttaaccaaaacactgaggataacgccaggatctcaatcacattctttcggctgttccgcgtgatgaggctggtgaagctcctgtctcggggagagggtattcggacgttgctctggaccttcattaaatcctttcaggctcttccctatgttgcattgctgatcgtaatgctgttcttcatttatgccgtcattgggatgcagatgtttggcaaaatcgccctgagggacaacagtcagatcaaccgaaacaacaactttcagacatttcctcaggctgttctgcttctcttcaggtgtgcgacaggagaggcgtggcaggaaatcatgctggcctgttctccaaaccgcccttgtgagaaggggtcagagatcaaccattccagcgaagactgtggcagtcactttgccatcttctactttgttagcttttatatgctttgcgccttcctgatcattaacctttttgtggctgtcatcatggacaactttgactatttaacacgggattggtcgatactgggaccgcatcatctagatgagttcaaaagaatatgggcagaatatgatcctgaggctaagggtcggataaagcatctggatgtagtcacgttactgcgcagaattcagccacctcttggatttggaaagctttgtccacatagagtggcatgcaagcgacttgtgtccatgaacatgcctctcaatagtgatggaacggtgatgtttaatgcaactctctttgccctggtacgaacggctctacgcatcgaaaccgaaggtaacctggagcaggcgaatgaggaacttagggccatcgtgaagaagatttggaagaggacaagcatgaagctgctggatcaagttgttccccctgctggagatgatgaagtcacagtggggaagttctatgctacattcctaattcaggaatacttcaggaaattcaagaagcgcaaggaacagggcctggtggccaaaatacccccaaagactgctctttcactgcaggctggcctgcgaacactacatgatatgggtccagagatcagaagagcgatatcaggagacttgaccgtggaggaggagctggaaagggccatgaaggaaactgtgtgtgctgcttctgaggatgatatcttcaggcggtctggtggcctcttcggtaaccatgtaaactactaccatcagagtgatggccacgtctccttcccacagtccttcaccacacagcggccattgcacatcagtaaatccgggagtcccggcgaggctgaatctccctctcatcagaagctggtggactccaccttcactcccagcagttactcttcctcaggatccaacgccaacatcaacaacgccaacaacaccgccatcggacaccgctatcccaaacctactgtcagcacggtggacggacaaacagggcctcctctcaccaccatcccactgccacgacccacatggtgctttcctaacaagagctctgattccagtgacagtcgtctccctataatacggcgagaggaagcatctacagatgagacatatgatgaaacatttttagatgagagagatcaggccatgctctccatggacatgctggaatttcaggatgaagaaagcaagcaactggctcctatggtggaggcagaagtaggggaggagagaaggccgtggcagtctccacggcgacgggccttcctctgccctactgcactgggtcgacggtcttcctttcacttggagtgtctgagaaagcataacagacctgacgtttctcagaagacagcactaccattgcacctagtgcatcatcaggctctggccgtggcagggttaagtcccttgctgagacggagtcactctccgacactgttcacacgtctgtgttccacacctcctgcgagtcccagtggccgcagcggtggaggcccatgctatcagcctgtgccctctctgcggctggagggcagcggatcttacgagaagctcaacagcagcatgccgtctgtgaactgcagctcatggtacagcgacagcaacggcaaccacagcgggagggcgcagcgacccgtgtcccttacagttcctccggtcacgcgcagagactctatatccctggcacacgggagcgccggcagcctcgtggaggcggtattgatatctgagggtttgggtcgctatgcacatgatccctccttcatccaggtagcgaagcaggaaatagcggaggcctgtgacatgacaatggaggagatggagaacgcggcagacaacattctcaacgctaacgcaccacccaatgctaacggaaacctgcttcccttcatacagtgcagagacactggctcgcaggagtcccgttgcagcctctcactgggcctttctcccgccacaggctctgatggggcactggaggcggagctagaggagtcagagggggcggggcagcggaacagccctctgatggaggatgaggacatggagtgcgtcacgagtttgtagggggaacagcagcagcacggaggacgtcctgtgtattagcccagctctctctgaggacactgctgtcctgcagtctgacctgtctgttgcgtgtacctataggggtataaggccattctgcttagctgagaccgagttggtttttagtttgtccgtgtgtttgcgagagcacgagcgagtaggtgtgtgtacatgtttgtgtgcgtgtttctaaaagtgcctcacagttctcacaaccttgattgcaaaaaaaaaaaaaaaaaaaaaaa

SEQ ID NO: 4 - protein, 2151 aa, Danio rerio
MVNESKNMYIPEDTLENHQGSNYSSPSLAPVPSLNEDEHVGGGGGVLGLAPKHIPTPGAPLSWQAAIFAARQAKLMGTTGAPISTASSTQRKRQHYTKPKKQASTASTRPPRALLCLTLKNPIRRACINIVEWKPFEIIILMTIFANCVALAVYIPFPEDDSNATNSNLERVEYGFLIIFTVEAFLKVIAYGLLCHPNAYLRNGWNLLDFIIVVVGLFSAILEQATKGDGGTSMGGKAAGFDVKALRAFRVLRPLRLVSGVPSLQVVLNSIIKAMVPLLHIALLVLFVIIIYAIIGLELFMGKMHRTGFFYKDGHKGHIAEEKPAPCAPSSAHGRHCSPPNITQCMMGWEGPNDGITNFDNFAFAMLTVFQCITMEGWTDVLYWMQDAMGYELPWVYFVSLVIFGSFFVLNLVLGVLSGEFSKEREKAKARGDFQKLREKQQLEEDLKGYLDWITQAEDIDPENDDEGLDDDKPRNLSMPASENESVNTDNAPAGDMEGETCCTRMANRISKSKFSRYSRRWNRLCRRKCRAAVKSNVFYWLVIFLVFLNTLTIASEHHQQPEWLTNVQDIANKVLLALFTGEMLLKMYSLGLQAYFVSLFNRFDSFVVCGGILETILVETKIMSPLGISVLRCVRLLRIFKITRYWNSLSNLVASLLNSVRSIASLLLLLFLFIIIFSLLGMQLFGGKFNFDETRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKRQNSEKPPLEDEKKEEKIELKSITSDGETPTATKINIDEYTGEDNEEKNPYPVNDFPAREDDDEEEPEMPGGPPPPPLSDIQLKEKDAPMPQDKTFFIFSPSNNFRVLWHKIVNHNIFTNLILLFILLSSISLPAEDPVKNDSFRNQILGYADYVFTGIFTIEIILKMTAYGAFLHKGSFCRNYFNILDLVVVSVSLISSGIQSSAINVVKILRVLRVLRPLRAINRAKGLKHVVQCVFVAIRTIGNIVIVTSLLQFMFACIGVQLLKGKFFYCTDTSKQTQAECRGAYILYKDGNVGEPEKAQRSWENSDFNFDDVLQGMMALFAVSTFEGWPGLLYRAIDSHAEDVGPIYNYRVVISIFFIIYIIIIAFFMMNIFVGFVIVTFQEQGEQEYKNCELDKNQRQCVEYALKARPLRRYIPKNPYQYKVWYVVNSTYFEYLMFTLILLNTICLAMQHHGQSQSFNKAMNILNMLFTGLFTVEMILKLIAFKPRHYFVDAWNTFDALIVVGSVVDIAITEVNQNTEDNARISITFFRLFRVMRLVKLLSRGEGIRTLLWTFIKSFQALPYVALLIVMLFFIYAVIGMQMFGKIALRDNSQINRNNNFQTFPQAVLLLFRCATGEAWQEIMLACSPNRPCEKGSEINHSSEDCGSHFAIFYFVSFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIETEGNLEQANEELRAIVKKIWKRTSMKLLDQVVPPAGDDEVTVGKFYATFLIQEYFRKFKKRKEQGLVAKIPPKTALSLQAGLRTLHDMGPEIRRAISGDLTVEEELERAMKETVCAASEDDIFRRSGGLFGNHVNYYHQSDGHVSFPQSFTTQRPLHISKSGSPGEAESPSHQKLVDSTFTPSSYSSSGSNANINNANNTAIGHRYPKPTVSTVDGQTGPPLTTIPLPRPTWCFPNKSSDSSDSRLPIIRREEASTDETYDETFLDERDQAMLSMDMLEFQDEESKQLAPMVEAEVGEERRPWQSPRRRAFLCPTALGRRSSFHLECLRKHNRPDVSQKTALPLHLVHHQALAVAGLSPLLRRSHSPTLFTRLCSTPPASPSGRSGGGPCYQPVPSLRLEGSGSYEKLNSSMPSVNCSSWYSDSNGNHSGRAQRPVSLTVPPVTRRDSISLAHGSAGSLVEAVLISEGLGRYAHDPSFIQVAKQEIAEACDMTMEEMENAADNILNANAPPNANGNLLPFIQCRDTGSQESRCSLSLGLSPATGSDGALEAELEESEGAGQRNSPLMEDEDMECVTSL

(19) United States
(12) Patent Application Publication
(10) Pub. No.: US 2004/0170993 A1
(43) Pub. Date: Sep. 2, 2004

(54) Methods for diagnosing and treating heart disease

(76) Inventors: Mark C. Fishman, Newton Center, MA (US); Wolfgang Rottbauer, Regensburg (DE)

Correspondence Address: CLARK & ELBING LLP, 101 FEDERAL STREET, BOSTON, MA, 02110, US

(21) Appl. No.: 10/467,491

(22) PCT Filed: Feb. 6, 2002
(86) PCT No.: PCT/US02/03441

Related U.S. Application Data
(60) Provisional application No. 60/267,332, filed on Feb. 7, 2001.

(51) Int. Cl. C12Q 1/68

(52) U.S. Cl. 435/6

(57) ABSTRACT

The invention provides methods of diagnosing heart disease, such as cardiac arrhythmia, methods for identifying compounds that can be used to treat or to prevent heart disease, and methods of using these compounds to treat or to prevent heart disease. Also provided in the invention are animal model systems that can be used in screening methods.

### FIELD OF THE INVENTION

This invention relates to methods for diagnosing and treating heart disease.

### BACKGROUND OF THE INVENTION

In humans, heart rhythm disturbances (i.e., cardiac arrythmias or tachyarrhythmias) are a common cause of morbidity and mortality. Arrhythmias that affect the atria fall into three main classes: atrial fibrillation, paroxysmal supraventricular tachycardia, and atrial flutter, and of these, atrial fibrillation is the most common. Indeed, in the United States, approximately two million patients are currently diagnosed with atrial fibrillation (Nattel et al., Annu. Rev. Physiol. 62:51-77, 2000). In this form of arrhythmia, the regular pumping action of the atria is replaced by disorganized, ineffective quivering, which prevents the heart from supplying an adequate amount of blood to the body. In rare cases, the cause of atrial fibrillation is hereditary, but usually the cause is unknown. However, atrial fibrillation is often associated with heart failure, rheumatic heart disease, coronary artery disease, left ventricular hypertrophy, cardiomyopathy, hypertension, and surgery, and may leave affected patients at high risk for stroke (Brugada et al., N. Engl. J. Med. 336:905-911, 1997; Gruver et al., Am. J. Cardiol. 83:13H-18H, 1999; Ryder et al., Am. J. Cardiol. 84:131R-138R, 1999).

Ion-conducting pores, such as calcium channels, play pivotal roles in the normal physiological functioning of the heart. Voltage-gated calcium channels, for example, mediate depolarization-induced influx of calcium ions across the plasma membrane of cardiac muscle, and thereby couple depolarization to contraction.

Cardiac heteromeric L-type calcium channels consist of several subunits. The α1 subunit forms the Ca⁺⁺ conducting pore of the channel, and consists of four transmembrane domains (I-IV), each of which is composed of six transmembrane segments (S 1-S6), including a highly charged amphipathic segment (S4), which may act as a voltage sensor for activation (Striessnig, Cell Physiol. Biochem. 9:242-269, 1999; Lehmann-Horn et al., Physiol. Rev. 79:1317-1372, 1999) (FIG. 1A). At least four classes of α1 subunits (α1C, α1D, α1F, and α1S), encoded by four different genes, conduct L-type calcium currents in humans. Cardiac myocytes express only the α1C L-type calcium channel subunit (Mikami et al., Nature 340:230-233, 1989; Welling et al., Circ. Res. 81:526-532, 1997). Furthermore, the α1C subunit is known to undergo alternative splicing, leading to the generation of three isoforms (α1C-A, α1C—B, and α1C—C), of which α1C-A is the predominant isoform expressed in the heart.

### SUMMARY OF THE INVENTION

The invention provides diagnostic, drug screening, and therapeutic methods that are based on the observation that a mutation in the α1C subunit of the voltage-dependent L-type calcium channel gene leads to a phenotype in zebrafish that is similar to a mammalian cardiac arrythmia, atrial fibrillation.

The invention provides a method of determining whether a test subject (e.g., a mammal, such as a human) has, or is at risk of developing, a disease or condition related to an α1C subunit of a voltage-dependent L-type calcium channel (e.g., heart disease, such as cardiac arrhythmia (e.g., atrial fibrillation). This method involves analyzing a nucleic acid molecule of a sample from the test subject to determine whether the test subject has a mutation (e.g., the island beat mutation) in a gene encoding the subunit. The presence of a mutation indicates that the test subject has, or is at risk of developing, a disease related to an α1C subunit of a voltage-dependent L-type calcium channel.

This method can further include the step of using nucleic acid molecule primers specific for a gene encoding the α1C subunit of a voltage-dependent L-type calcium channel for nucleic acid molecule amplification of the gene by the polymerase chain reaction. Determination of whether the gene includes a mutation can be carried out by sequencing a nucleic acid molecule encoding an α1C subunit of a voltage-dependent L-type calcium channel from the subject.

In a second aspect, the invention provides a method for identifying a compound that can be used to treat or to prevent heart disease (e.g., a cardiac arrhythmia (e.g., atrial fibrillation). This method involves contacting an organism (e.g., a zebrafish) having a mutation (e.g., the island beat mutation) in a gene encoding an α1C subunit of a voltage-dependent L-type calcium channel and having a phenotype characteristic of heart disease with the compound, and determining the effect of the compound on the phenotype. Detection of an improvement in the phenotype indicates the identification of a compound that can be used to treat or to prevent heart disease.

In a third aspect, the invention provides a method of treating or preventing heart disease (e.g., a cardiac arrhythmia (e.g., atrial fibrillation)) in a patient, involving administering to the patient a compound identified using the method described above. The patient may have, e.g., a mutation in a gene encoding an α1C subunit of a voltage-dependent L-type calcium channel, such as the island beat mutation.

In a fourth aspect, the invention provides a method of treating or preventing heart disease in a patient, involving administering to the patient a functional α1C subunit of a voltage-dependent L-type calcium channel or an expression vector including a nucleic acid molecule encoding this subunit.

In a fifth aspect, the invention provides a substantially pure zebrafish α1C subunit of a voltage-dependent L-type calcium channel. This polypeptide can include or consist essentially of an amino acid sequence that is substantially identical to the amino acid sequence of SEQ ID NO:2.

In a sixth aspect, the invention provides a substantially pure nucleic acid molecule (e.g., a DNA molecule) including a sequence encoding a zebrafish α1C subunit of a voltage-dependent L-type calcium channel. This nucleic acid molecule can encode a polypeptide having an amino sequence that is substantially identical to the amino acid sequence of SEQ ID NO:2.

In a seventh aspect, the invention includes the use of a compound identified using the method described above in the preparation of a medicament for treating or preventing heart disease in a patient.

In an eighth aspect, the invention includes the use of a ctC subunit of a voltage-dependent L-type calcium channel or an expression vector including a nucleic acid molecule encoding said subunit in the preparation of a medicament for treating or preventing heart disease in a patient.

In further aspects, the invention includes a vector including the nucleic acid molecule described above, a cell including this vector, a non-human transgenic animal (e.g., a zebrafish) including the nucleic acid molecule described above, a non-human animal having a knockout mutation in one or both alleles encoding a α1C subunit polypeptide, a cell from this animal, a non-human transgenic animal (e.g., a zebrafish) including a nucleic acid molecule encoding a mutant α1C subunit of a voltage-dependent L-type calcium channel (e.g., an island beat mutant), and an antibody that specifically binds to an α1C subunit of a voltage-dependent L-type calcium channel.

By “polypeptide” or “polypeptide fragment” is meant a chain of two or more amino acids, regardless of any post-translational modification (e.g., glycosylation or phosphorylation), constituting all or part of a naturally or non-naturally occurring polypeptide. By “post-translational modification” is meant any change to a polypeptide or polypeptide fragment during or after synthesis. Post-translational modifications can be produced naturally (such as during synthesis within a cell) or generated artificially (such as by recombinant or chemical means). A “protein” can be made up of one or more polypeptides.

By “α1C subunit,” “α1C subunit protein,” or “α1C subunit polypeptide” is meant a polypeptide that has at least 45%, preferably at least 60%, more preferably at least 75%, and most preferably at least 90% amino acid sequence identity to the sequence of the human (see, e.g., SEQ ID NO:2) or the zebrafish (see, e.g., SEQ ID NO:4) α1C subunit of voltage-dependent L-type calcium channels. Polypeptide products from splice variants of α1C subunit gene sequences and α1C subunit genes containing mutations are also included in this definition. An α1C subunit polypeptide as defined herein plays a role in heart development, modeling, and function. It can be used as a marker of heart disease, such as cardiac arrythmia, e.g., atrial fibrillation. The invention thus includes proteins having any of these and other functions of α1C subunit polypeptides, as described herein, and having sequence identity (e.g., at least 75%, 85%, 90%, or 95%) to a human (SEQ ID NO:2) or a zebrafish (SEQ ID NO:4) α1C subunit polypeptide.

By an “α1C subunit nucleic acid molecule” is meant a nucleic acid molecule, such as a genomic DNA, cDNA, or RNA (e.g., mRNA) molecule, that encodes an α1C subunit (e.g., a human (SEQ ID NO: 1) or a zebrafish (SEQ ID NO:3) α1C subunit), an α1C subunit protein, an α1C subunit polypeptide, or a portion thereof, as defined above. A mutation in an α1C subunit nucleic acid molecule can be characterized, for example, by a C to T nucleotide transversion at the first base of codon 1077 (CAG->TAG), predicting a change from glutamine to a stop codon, or a T to A nucleotide transversion in codon 1352 (TTG->TAG, which changes a leucine to a stop codon. In addition to these zebrafish island beat mutations, the invention includes any mutation that results in aberrant α1C subunit subunit production or function, including, only as examples, null mutations and mutations causing truncations.

The term “identity” is used herein to describe the relationship of the sequence of a particular nucleic acid molecule or polypeptide to the sequence of a reference molecule of the same type. For example, if a polypeptide or a nucleic acid molecule has the same amino acid or nucleotide residue at a given position, compared to a reference molecule to which it is aligned, there is said to be “identity” at that position. The level of sequence identity of a nucleic acid molecule or a polypeptide to a reference molecule is typically measured using sequence analysis software with the default parameters specified therein, such as the introduction of gaps to achieve an optimal alignment (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). These software programs match identical or similar sequences by assigning degrees of identity to various substitutions, deletions, or other modifications. Conservative substitutions typically include substitutions within the following groups: glycine, alanine, valine, isoleucine, and leucine; aspartic acid, glutamic acid, asparagine, and glutamine; serine and threonine; lysine and arginine; and phenylalanine and tyrosine.

A nucleic acid molecule or polypeptide is said to be “substantially identical” to a reference molecule if it exhibits, over its entire length, at least 51%, preferably at least 55%, 60%, or 65%, and most preferably 75%, 85%, 90%, or 95% identity to the sequence of the reference molecule. For polypeptides, the length of comparison sequences is at least 16 amino acids, preferably at least 20 amino acids, more preferably at least 25 amino acids, and most preferably at least 35 amino acids. For nucleic acid molecules, the length of comparison sequences is at least 50 nucleotides, preferably at least 60 nucleotides, more preferably at least 75 nucleotides, and most preferably at least 110 nucleotides.

An α1C subunit nucleic acid molecule or an α1C subunit polypeptide is “analyzed” or subject to “analysis” if a test procedure is carried out on it that allows the determination of its biological activity or whether it is wild type or mutated. For example, one can analyze the α1C subunit genes of an animal (e.g., a human or a zebrafish) by amplifying genomic DNA of the animal using the polymerase chain reaction, and then determining whether the amplified DNA contains a mutation, for example, the island beat mutation, by, e.g., nucleotide sequence or restriction fragment analysis.

By “probe” or “primer” is meant a single-stranded DNA or RNA molecule of defined sequence that can base pair to a second DNA or RNA molecule that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes or primers specific for α1C subunit nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to human (SEQ ID NO: 1) or zebrafish (SEQ ID NO:3) gene sequences encoding the α1C subunit.

Probes can be detectably-labeled, either radioactively or non-radioactively, by methods that are well-known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

A molecule, e.g., an oligonucleotide probe or primer, a gene or fragment thereof, a cDNA molecule, a polypeptide, or an antibody, can be said to be “detectably-labeled” if it is marked in such a way that its presence can be directly identified in a sample. Methods for detectably-labeling molecules are well known in the art and include, without limitation, radioactive labeling (e.g., with an isotope, such as ³²P or ³⁵S) and nonradioactive labeling (e.g., with a fluorescent label, such as fluorescein).

By a “substantially pure polypeptide” is meant a polypeptide (or a fragment thereof) that has been separated from proteins and organic molecules that naturally accompany it. Typically, a polypeptide is substantially pure when it is at least 60%, by weight, free from the proteins and naturally-occurring organic molecules with which it is naturally associated. Preferably, the polypeptide is an α1C subunit polypeptide that is at least 75%, more preferably at least 90%, and most preferably at least 99%, by weight, pure. A substantially pure α1C subunit polypeptide can be obtained, for example, by extraction from a natural source (e.g., isolated heart tissue), by expression of a recombinant nucleic acid molecule encoding an α1C subunit polypeptide, or by chemical synthesis. Purity can be measured by any appropriate method, e.g., by column chromatography, polyacrylamide gel electrophoresis, or HPLC analysis.

A polypeptide is substantially free of naturally associated components when it is separated from those proteins and organic molecules that accompany it in its natural state. Thus, a protein that is chemically synthesized or produced in a cellular system that is different from the cell in which it is naturally produced is substantially free from its naturally associated components. Accordingly, substantially pure polypeptides not only include those that are derived from eukaryotic organisms, but also those synthesized in E. coli or other prokaryotes.

An antibody is said to “specifically bind” to a polypeptide if it recognizes and binds to the polypeptide (e.g., an α1C subunit polypeptide), but does not substantially recognize and bind to other molecules (e.g., non-α1C subunit related polypeptides) in a sample, e.g., a biological sample that naturally includes the polypeptide.

By “high stringency conditions” is meant conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1× Denhardt's solution, 10% dextran sulfate, and 0.1% SDS, at a temperature of 42° C. (These are typical conditions for high stringency northern or Southern hybridizations.) High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually 16 nucleotides or longer for PCR or sequencing, and 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

By “sample” is meant a tissue biopsy, amniotic fluid, cell, blood, serum, urine, stool, or other specimen obtained from a patient or a test subject. The sample can be analyzed to detect a mutation in an α1C subunit gene, or expression levels of an α1C subunit gene, by methods that are known in the art. For example, methods such as sequencing, single-strand conformational polymorphism (SSCP) analysis, or restriction fragment length polymorphism (RFLP) analysis of PCR products derived from a patient sample can be used to detect a mutation in an α1C subunit gene; ELISA can be used to measure levels of an α1C subunit polypeptide; and PCR can be used to measure the level of an α1C subunit nucleic acid molecule.

By “α1C subunit-related disease” or “α1C subunit-related condition” is meant a disease or condition that results from inappropriately high or low expression of an α1C subunit gene, or a mutation in an α1C subunit gene that alters the biological activity of an α1C subunit nucleic acid molecule or polypeptide. α1C subunit-related diseases and conditions can arise in any tissue in which an α1C subunit is expressed during prenatal or post-natal life. α1C subunit-related diseases and conditions can include heart diseases, such as cardiac arrythmia (e.g., atrial fibrillation).

The invention provides several advantages. For example, using the diagnostic methods of the invention, it is possible to detect an increased likelihood of heart disease, such as cardiac arrythmia (e.g., atrial fibrillation), in a patient, so that appropriate intervention can be instituted before any symptoms occur. This may be useful, for example, with patients in high risk groups for cardiac arrythmia (e.g., atrial fibrillation; see above). Also, the diagnostic methods of the invention facilitate determination of the etiology of an existing heart condition, such as a cardiac arrythmia, in a patient so that an appropriate approach to treatment can be selected. In addition, the screening methods of the invention can be used to identify compounds that can be used to treat or to prevent heart conditions, such as cardiac arrythmia (e.g., atrial fibrillation).

Other features and advantages of the invention will be apparent from the following detailed description, the drawings, and the claims.

### BRIEF DESCRIPTION OF THE DRAWINGS

FIGS. 1A and 1B are diagrams showing the domain structure of the L-type calcium channel α1 subunit (FIG. 1A), partial sequence alignment of zebrafish and human isl proteins (FIG. 1B), and the positions of the isl mutations (FIGS. 1A and 1B). Domains and segments are indicated above the alignment. Black boxes signify amino acid identity, and isl mutation sites are indicated by asterisks.

FIG. 2 is a diagram of the zebrafish genomic region including the L-type calcium channel α1 subunit gene.

### DETAILED DESCRIPTION

The invention provides methods of diagnosing heart disease, screening methods for identifying compounds that can be used to treat or to prevent heart disease, and methods of treating or preventing heart disease using such compounds. In particular, we have discovered that a mutation (the island beat mutation) in a gene encoding the α1C subunit of the zebrafish voltage-dependent L-type calcium channel leads to a phenotype in zebrafish that is similar to a human cardiac arrhythmia, atrial fibrillation. Thus, the diagnostic methods of the invention involve detection of mutations in genes encoding the α1C subunit of voltage-dependent L-type calcium channels, including mutations affecting the predominant isoform expressed in the heart (α1C-A), while the compound identification methods involve screening for compounds that affect the phenotype of organisms having mutations in genes encoding such subunits of these channels or other models of cardiac arrhythmia. Compounds identified in this manner can be used in methods to treat or to prevent heart disease, such as cardiac arrhythmia (e.g., atrial fibrillation).

The invention also provides animal model systems (e.g., zebrafish having mutations (e.g., the island beat mutations) in genes encoding the α1C subunit of voltage-dependent L-type calcium channels, or mice (or other animals) having such mutations) that can be used in the screening methods mentioned above, as well as the α1C subunit of the zebrafish voltage-dependent L-type calcium channel, and genes encoding this protein. Also included in the invention are genes encoding mutant zebrafish α1C subunits (e.g., genes having the island beat mutation) and proteins encoded by these genes. Antibodies that specifically bind to these proteins (wild type or mutant) are also included in the invention.

The diagnostic, screening, and therapeutic methods of the invention, as well as the animal model systems, proteins, and genes of the invention, are described further, as follows.

Diagnostic Methods

Nucleic acid molecules encoding the α1C subunit of voltage-dependent L-type calcium channels (e.g., the α1C-A isoform), as well as polypeptides encoded by these nucleic acid molecules and antibodies specific for these polypeptides, can be used in methods to diagnose or to monitor diseases and conditions involving mutations in, or inappropriate expression of, genes encoding this subunit. As discussed above, the island beat mutation in zebrafish, which is present in a gene encoding the α1C subunit of a voltage-dependent L-type calcium channel, is characterized by a phenotype that is similar to that of atrial fibrillation in humans. Thus, detection of abnormalities in genes encoding the α1C subunit of voltage-dependent L-type calcium channels or in their expression can be used in methods to diagnose, or to monitor treatment or development of, human heart disease, such as cardiac arrhythmias (e.g., atrial fibrillation).

The diagnostic methods of the invention can be used, for example, with patients that have cardiac arrhythmia, in an effort to determine its etiology and, thus, to facilitate selection of an appropriate course of treatment. The diagnostic methods can also be used with patients who have not yet developed cardiac arrhythmia, but who are at risk of developing such a disease, or with patients that are at an early stage of developing such a disease. Also, the diagnostic methods of the invention can be used in prenatal genetic screening, for example, to identify parents who may be carriers of a recessive mutation in a gene encoding the a α1C subunit of a voltage-dependent L-type calcium channel.

Examples of cardiac arrhythmias that can be diagnosed and treated using the methods of the invention are atrial fibrillation, atrial flutter, and paroxysmal supraventricular tachycardia, all of which are rhythm disorders of the atria. Atrial fibrillation is often associated with other forms of cardiovascular disease which, thus, can also be diagnosed using the methods of the invention. These diseases include, for example, congestive heart failure, which is characterized by accumulation of excess fluid in the lungs and body; rheumatic heart disease, which is caused by permanent damage to heart valves; coronary heart disease, in which blood flow through the coronary arteries of the heart muscle is reduced; left ventricular hypertrophy; cardiomyopathy, which can either be characterized by dilation of the heart and concurrent thinning of the heart walls, or by hypertrophy of cardiac muscle, enlargement of the heart, rigidity and loss of flexibility of the heart walls, and narrowing of the ventricular cavities; and hypertension (i.e., high blood pressure). The methods of the invention can be used to diagnose or to treat the disorders described herein in any mammal, for example, in humans, domestic pets, or livestock.

Abnormalities in an α1C subunit of a voltage-dependent L-type calcium channel that can be detected using the diagnostic methods of the invention include those characterized by, for example, (i) a gene encoding an α1C subunit containing a mutation that results in the production of an abnormal α1C subunit polypeptide, (ii) an abnormal α1C subunit polypeptide itself, and (iii) a mutation in a gene encoding an α1C subunit of a voltage-dependent L-type calcium channel that results in production of an abnormal amount of this subunit. Detection of such abnormalities can be used in methods to diagnose human heart disease, such as cardiac arrhythmia (e.g., atrial fibrillation). Exemplary of the mutations in an α1C subunit of a voltage-dependent L-type calcium channel that can be detected using the methods of the invention is the island beat mutation (see below).

A mutation in a gene encoding an α1C subunit of a voltage-dependent L-type calcium channel can be detected in any tissue of a subject, even one in which this subunit is not expressed. Because of the limited number of tissues in which these channels are expressed (i.e., the myocardium, neurons, and smooth muscle) and because of the undesirability of sampling such tissues for assays, it may be preferable to detect mutant genes in other, more easily obtained sample types, such as in blood or amniotic fluid samples.

Detection of a mutation in a gene encoding the α1C subunit of a voltage-dependent L-type calcium channel can be carried out using any standard diagnostic technique. For example, a biological sample obtained from a patient can be analyzed for one or more mutations (e.g., an island beat mutation; see below) in nucleic acid molecules encoding an α1C subunit using a mismatch detection approach. Generally, this approach involves polymerase chain reaction (PCR) amplification of nucleic acid molecules from a patient sample, followed by identification of a mutation (i.e., a mismatch) by detection of altered hybridization, aberrant electrophoretic gel migration, binding, or cleavage mediated by mismatch binding proteins, or by direct nucleic acid molecule sequencing. Any of these techniques can be used to facilitate detection of a mutant gene encoding an a α1C subunit of a voltage-dependent L-type calcium channel, and each is well known in the art. For instance, examples of these techniques are described by Orita et al. (Proc. Natl. Acad. Sci. U.S.A. 86:2766-2770, 1989) and Sheffield et al. (Proc. Natl. Acad. Sci. U.S.A. 86:232-236, 1989).

In addition to facilitating diagnosis of existing heart disease, mutation detection assays also provide an opportunity to diagnose a predisposition to heart disease related to a mutation in a gene encoding the α1C subunit of a voltage-dependent L-type calcium channel before the onset of symptoms. For example, a patient who is heterozygous for a gene encoding an abnormal α1C subunit of a voltage-dependent L-type calcium channel (or an abnormal amount thereof) that suppresses normal voltage-dependent L-type calcium channel biological activity or expression may show no clinical symptoms of a disease related to such channels, and yet possess a higher than normal probability of developing heart disease, such as cardiac arrhythmia (e.g., atrial fibrillation). Given such a diagnosis, a patient can take precautions to minimize exposure to adverse environmental factors, and can carefully monitor their medical condition, for example, through frequent physical examinations. As mentioned above, this type of diagnostic approach can also be used to detect a mutation in a gene encoding the α1C subunit of voltage-dependent L-type calcium channels in prenatal screens.

While it may be preferable to carry out diagnostic methods for detecting a mutation in a gene encoding the α1C subunit of a voltage-dependent L-type calcium channel using genomic DNA from readily accessible tissues, mRNA encoding this subunit, or the subunit itself, can also be assayed from tissue samples in which it is expressed, and may not be so readily accessible. For example, expression levels of a gene encoding the α1C subunit of a voltage-dependent L-type calcium channel in such a tissue sample from a patient can be determined by using any of a number of standard techniques that are well known in the art, including northern blot analysis and quantitative PCR (see, e.g., Ausubel et al., supra; PCR Technology: Principles and Applications for DNA Amplification, H. A. Ehrlich, Ed., Stockton Press, NY; Yap et al. Nucl. Acids. Res. 19:4294, 1991).

In another diagnostic approach of the invention, an immunoassay is used to detect or to monitor the level of an α1C subunit protein in a biological sample. Polyclonal or monoclonal antibodies specific for the α1C subunit of a voltage-dependent L-type calcium channel, e.g., antibodies specific for the α1C-A subunit, can be used in any standard immunoassay format (e.g., ELISA, Western blot, or RIA; see, e.g., Ausubel et al., supra) to measure polypeptide levels of the α1C subunit. These levels can be compared to levels of the α1C subunit in a sample from an unaffected individual. Detection of a decrease in production of the α1C subunit using this method, for example, may be indicative of a condition or a predisposition to a condition involving insufficient biological activity of the α1C subunit of voltage-dependent L-type calcium channels.

Immunohistochemical techniques can also be utilized for detection of the α1C subunit of voltage-dependent L-type calcium channels in patient samples. For example, a tissue sample can be obtained from a patient, sectioned, and stained for the presence of the α1C subunit of a voltage-dependent L-type calcium channel using an anti-α1C subunit or anti-α1C-A subunit antibody and any standard detection system (e.g., one that includes a secondary antibody conjugated to an enzyme such as horseradish peroxidase). General guidance regarding such techniques can be found in, e.g., Bancroft et al., Theory and Practice of Histological Techniques, Churchill Livingstone, 1982, and Ausubel et al., supra.

Identification of Molecules that can be used to Treat or to Prevent Cardiac Arrhythmia

Identification of a mutation in a gene encoding the α1C subunit of a voltage-dependent L-type calcium channel as resulting in a phenotype that is related to cardiac arrhythmia facilitates the identification of molecules (e.g., small organic or inorganic molecules, peptides, or nucleic acid molecules) that can be used to treat or to prevent heart disease, such as cardiac arrhythmia (e.g., atrial fibrillation). The effects of candidate compounds on cardiac arrhythmia can be investigated using, for example, the zebrafish system. The zebrafish, Danio rerio, is a convenient organism to use in genetic analysis of vascular development. In addition to its short generation time and fecundity, it has an accessible and transparent embryo, allowing direct observation of blood vessel function from the earliest stages of development. As discussed further below, zebrafish and other animals having a mutation (e.g., the island beat mutation) in a gene encoding the a α1C subunit of a voltage-dependent L-type calcium channel, which can be used in these methods, are also included in the invention.

In one example of the screening methods of the invention, a zebrafish having a mutation in a gene encoding the α1C subunit of a voltage-dependent L-type calcium channel (e.g., a zebrafish having the island beat mutation) is contacted with a candidate compound, and the effect of the compound on the development of a heart abnormality that is characteristic of cardiac arrhythmia, or on the status of such an existing heart abnormality, is monitored relative to an untreated, identically mutant control. As discussed further below, zebrafish having the island beat mutation are characterized by a lack of peristaltic movement of the heart tube, independent of individual atrial cardiomyocyte contraction, a collapsed and silent ventricle, and a dilated and thickened atrium. Thus, these characteristics (in addition to other characteristics of heart disease) can be monitored using the screening methods of the invention.

After a compound has been shown to have a desired effect in the zebrafish system, it can be tested in other models of heart disease, for example, in mice or other animals having a mutation in a gene encoding the α1C subunit of voltage-dependent L-type calcium channels. Alternatively, testing in such animal model systems can be carried out in the absence of zebrafish testing.

Cell culture-based assays can also be used in the identification of molecules that increase or decrease α1C subunit levels or biological activity. According to one approach, candidate molecules are added at varying concentrations to the culture medium of cells expressing α1C subunit mRNA. α1C subunit biological activity is then measured using standard techniques. The measurement of biological activity can include the measurement of α1C subunit protein and nucleic acid molecule levels.

In general, novel drugs for prevention or treatment of heart diseases related to mutations in a gene encoding the α1C subunit of a voltage-dependent L-type calcium channel can be identified from large libraries of natural products, synthetic (or semi-synthetic) extracts, and chemical libraries using methods that are well known in the art. Those skilled in the field of drug discovery and development will understand that the precise source of test extracts or compounds is not critical to the screening methods of the invention and that dereplication, or the elimination of replicates or repeats of materials already known for their therapeutic activities for heart disease can be employed whenever possible.

Candidate compounds to be tested include purified (or substantially purified) molecules or one or more component of a mixture of compounds (e.g., an extract or supernatant obtained from cells; Ausubel et al., supra) and such compounds further include both naturally occurring or artificially derived chemicals and modifications of existing compounds. For example, candidate compounds can be polypeptides, synthesized organic or inorganic molecules, naturally occurring organic or inorganic molecules, nucleic acid molecules, and components thereof.

Numerous sources of naturally occurring candidate compounds are readily available to those skilled in the art. For example, naturally occurring compounds can be found in cell (including plant, fungal, prokaryotic, and animal) extracts, mammalian serum, growth medium in which mammalian cells have been cultured, protein expression libraries, or fermentation broths. In addition, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla.), and PharmaMar, U.S.A. (Cambridge, Mass.). Furthermore, libraries of natural compounds can be produced, if desired, according to methods that are known in the art, e.g., by standard extraction and fractionation.

Artificially derived candidate compounds are also readily available to those skilled in the art. Numerous methods are available for generating random or directed synthesis (e.g., semi-synthesis or total synthesis) of any number of chemical compounds, including, for example, saccharide-, lipid-, peptide-, and nucleic acid molecule-based compounds. In addition, synthetic compound libraries are commercially available from Brandon Associates (Merrimack, N.H.) and Aldrich Chemicals (Milwaukee, WI). Libraries of synthetic compounds can also be produced, if desired, according to methods known in the art, e.g., by standard extraction and fractionation. Furthermore, if desired, any library or compound can be readily modified using standard chemical, physical, or biochemical methods.

When a crude extract is found to have an effect on the development or persistence of heart disease, further fractionation of the positive lead extract can be carried out to isolate chemical constituents responsible for the observed effect. Thus, the goal of the extraction, fractionation, and purification process is the careful characterization and identification of a chemical entity within the crude extract having a desired activity. The same assays described herein for the detection of activities in mixtures of compounds can be used to purify the active component and to test derivatives of these compounds. Methods of fractionation and purification of such heterogeneous extracts are well known in the art. If desired, compounds shown to be useful agents for treatment can be chemically modified according to methods known in the art.

Animal Model Systems

The invention also provides animal model systems for use in carrying out the screening methods described above. Examples of these model systems include zebrafish and other animals, such as mice, that have a mutation (e.g., the island beat mutation) in a gene encoding an α1C subunit of voltage-dependent L-type calcium channel. For example, a zebrafish model that can be used in the invention can include a mutation that results in a lack of α1C subunit production or production of a truncated (e.g., by introduction of a stop codon) or otherwise altered α1C subunit gene product. As a specific example, a zebrafish having the island beat mutation can be used (see below).

Treatment or Prevention of Cardiac Arrhythmia

Compounds identified using the screening methods described above can be used to treat patients that have or are at risk of developing heart disease, such as cardiac arrhythmia (e.g., atrial fibrillation). Nucleic acid molecules encoding the α1C subunit of a voltage-dependent L-type calcium channel, as well as these channels themselves, can also be used in such methods. Treatment may be required only for a short period of time or may, in some form, be required throughout a patient's lifetime. Any continued need for treatment, however, can be determined using, for example, the diagnostic methods described above. In considering various therapies, it is to be understood that such therapies are, preferably, targeted to the affected or potentially affected organ (i.e., the heart).

Treatment or prevention of diseases resulting from a mutated gene encoding the α1C subunit of a voltage-dependent L-type calcium channel can be accomplished, for example, by modulating the function of a mutant α1C subunit protein or the channel in which it occurs. Treatment can also be accomplished by delivering normal α1C subunit protein to appropriate cells, altering the levels of normal or mutant α1C subunit protein, replacing a mutant gene encoding an α1C subunit with a normal gene encoding the α1C subunit, or administering a normal gene encoding the α1C subunit of a voltage-dependent L-type calcium channel. It is also possible to correct the effects of a defect in a gene encoding the α1C subunit of a voltage-dependent L-type calcium channel by modifying the physiological pathway (e.g., a signal transduction pathway) in which the α1C subunit of a voltage-dependent L-type calcium channel participates.

In a patient diagnosed as being heterozygous for a gene encoding a mutant α1C subunit of a voltage-dependent L-type calcium channel, or as susceptible to such mutations or aberrant α1C subunit expression (even if those mutations or expression patterns do not yet result in alterations in expression or biological activity of the α1C subunit), any of the therapies described herein can be administered before the occurrence of the disease phenotype. In particular, compounds shown to have an effect on the phenotype of α1C subunit mutants, or to modulate expression of α1C subunits can be administered to patients diagnosed with potential or actual heart disease by any standard dosage and route of administration.

Any appropriate route of administration can be employed to administer a compound found to be effective in treating or preventing cardiac arrhythmia according to the invention. For example, administration can be parenteral, intravenous, intra-arterial, subcutaneous, intramuscular, intraventricular, intracapsular, intraspinal, intracisternal, intraperitoneal, intranasal, by aerosol, by suppository, or oral.

A therapeutic compound of the invention can be administered within a pharmaceutically-acceptable diluent, carrier, or excipient, in unit dosage form. Administration can begin before or after the patient is symptomatic. Methods that are well known in the art for making formulations are found, for example, in Remington 's Pharmaceutical Sciences (18^{th} edition), ed. A. Gennaro, 1990, Mack Publishing Company, Easton, Pa. Therapeutic formulations can be in the form of liquid solutions or suspensions. Formulations for parenteral administration can, for example, contain excipients; sterile water; or saline; polyalkylene glycols, such as polyethylene glycol; oils of vegetable origin; or hydrogenated napthalenes. Biocompatible, biodegradable lactide polymer, lactide/glycolide copolymer, or polyoxyethylene-polyoxypropylene copolymers can be used to control the release of the compounds. Other potentially useful parenteral delivery systems for compounds identified using the methods of the invention include ethylene-vinyl acetate copolymer particles, osmotic pumps, implantable infusion systems, and liposomes. For oral administration, formulations can be in the form of tablets or capsules. Formulations for inhalation can contain excipients, for example, lactose, or can be aqueous solutions containing, for example, polyoxyethylene-9-lauryl ether, glycocholate, and deoxycholate, or can be oily solutions for administration in the form of nasal drops, or as a gel. Alternatively, intranasal formulations can be in the form of powders or aerosols.

To replace a mutant protein with normal protein, or to add protein to cells that do not express sufficient or normal α1C subunit protein, it may be necessary to obtain large amounts of pure α1C subunit protein from cultured cell systems in which the protein is expressed (see, e.g., below). Delivery of the protein to the affected tissue can then be accomplished using appropriate packaging or administration systems.

Gene therapy is another therapeutic approach for preventing or ameliorating diseases (e.g., cardiac arrythmias, such as atrial fibrillation) caused by α1C subunit gene defects. Nucleic acid molecules encoding wild type α1C subunits can be delivered to cells that lack sufficient, normal α1C subunit biological activity (e.g., cells carrying mutations (e.g., the island beat mutation) in α1C subunit genes). The nucleic acid molecules must be delivered to those cells in a form in which they can be taken up by the cells and so that sufficient levels of protein, to provide effective α1C subunit function, can be produced. Alternatively, for some α1C subunit mutations, it may be possible slow the progression of the resulting disease or to modulate a α1C subunit activity by introducing another copy of a homologous gene bearing a second mutation in that gene, to alter the mutation, or to use another gene to block any negative effect.

Transducing retroviral, adenoviral, and adeno-associated viral vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997).

For example, the full length α1C subunit gene, or a portion thereof, can be cloned into a retroviral vector and expression can be driven from its endogenous promoter, from the retroviral long terminal repeat, or from a promoter specific for a target cell type of interest (such as cardiac muscle or other vascular cells). Other viral vectors that can be used include, for example, vaccinia virus, bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the introduction of therapeutic DNA into cells predicted to be subject to diseases involving the α1C subunit. For example, an α1C subunit nucleic acid molecule or an antisense nucleic acid molecule can be introduced into a cell by lipofection (Felgner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990).

Gene transfer can also be achieved using non-viral means involving transfection in vitro. Such methods include use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal α1C subunit gene into a cultivatable cell type ex vivo, after which the cell (or its descendants) are injected into a targeted tissue.

α1C subunit cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct α1C subunit expression. The enhancers used can include, without limitation, those that are characterized as tissue- or cell-specific enhancers. Alternatively, if an α1C subunit genomic clone is used as a therapeutic construct (such clones can be identified by hybridization with α1C subunit cDNA, as described herein), regulation can be mediated by the cognate regulatory sequences or, if desired, by regulatory sequences derived from a heterologous source, including any of the promoters or regulatory elements described above.

Antisense-based strategies can be employed to explore α1C subunit gene function and as a basis for therapeutic drug design. These strategies are based on the principle that sequence-specific suppression of gene expression (via transcription or translation) can be achieved by intracellular hybridization between genomic DNA or mRNA and a complementary antisense species. The formation of a hybrid RNA duplex interferes with transcription of the target α1C subunit-encoding genomic DNA molecule, or processing, transport, translation, or stability of the target α1C subunit mRNA molecule.

Antisense strategies can be delivered by a variety of approaches. For example, antisense oligonucleotides or antisense RNA can be directly administered (e.g., by intravenous injection) to a subject in a form that allows uptake into cells.

Alternatively, viral or plasmid vectors that encode antisense RNA (or antisense RNA fragments) can be introduced into a cell in vivo or ex vivo. Antisense effects can be induced by control (sense) sequences; however, the extent of phenotypic changes is highly variable. Phenotypic effects induced by antisense effects are based on changes in criteria such as protein levels, protein activity measurement, and target mRNA levels.

α1C subunit gene therapy can also be accomplished by direct administration of antisense α1C subunit mRNA to a cell that is expected to be adversely affected by the expression of wild-type or mutant α1C subunit. The antisense α1C subunit mRNA can be produced and isolated by any standard technique, but is most readily produced by in vitro transcription using an antisense α1C subunit cDNA under the control of a high efficiency promoter (e.g., the T7 promoter). Administration of antisense α1C subunit mRNA to cells can be carried out by any of the methods for direct nucleic acid molecule administration described above.

An alternative strategy for inhibiting α1C subunit function using gene therapy involves intracellular expression of an anti-α1C subunit antibody or a portion of an anti-α1C subunit antibody. For example, the gene (or gene fragment) encoding a monoclonal antibody that specifically binds to an α1C subunit and inhibits its biological activity can be placed under the transcriptional control of a tissue-specific gene regulatory sequence.

Another therapeutic approach included in the invention involves administration of a recombinant α1C subunit polypeptide, either directly to the site of a potential or actual disease-affected tissue (for example, by injection) or systemically (for example, by any conventional recombinant protein administration technique). The dosage of the α1C subunit depends on a number of factors, including the size and health of the individual patient but, generally, between 0.1 mg and 100 mg, inclusive, is administered per day to an adult in any pharmaceutically acceptable formulation.

Synthesis of α1C Subunit Proteins, Polypeptides, and Polypeptide Fragments

Those skilled in the art of molecular biology will understand that a wide variety of expression systems can be used to produce the recombinant α1C subunit proteins. As discussed further below, the precise host cell used is not critical to the invention. The α1C subunit proteins can be produced in a prokaryotic host (e.g., E. coli ) or in a eukaryotic host (e.g., S. cerevisiae , insect cells such as Sf9 cells, or mammalian cells such as COS-1, NIH 3T3, or HeLa cells). These cells are commercially available from, for example, the American Type Culture Collection, Manassas, Va. (see also Ausubel et al., supra). The method of transformation and the choice of expression vehicle (e.g., expression vector) will depend on the host system selected. Transformation and transfection methods are described, e.g., in Ausubel et al., supra, and expression vehicles can be chosen from those provided, e.g., in Pouwels et al., Cloning Vectors: A Laboratory Manual, 1985, Supp. 1987. Specific examples of expression systems that can be used in the invention are described further, as follows.

For protein expression, eukaryotic or prokaryotic expression systems can be generated in which α1C subunit gene sequences are introduced into a plasmid or other vector, which is then used to transform living cells. Constructs in which full length α1C subunit cDNAs, containing the entire open reading frame, inserted in the correct orientation into an expression plasmid can be used for protein expression. Alternatively, portions of α1C subunit gene sequences, including wild type or mutant α1C subunit sequences, can be inserted. Prokaryotic and eukaryotic expression systems allow various important functional domains of α1C subunit proteins to be recovered, if desired, as fusion proteins, and then used for binding, structural, and functional studies, and also for the generation of antibodies.

Typical expression vectors contain promoters that direct synthesis of large amounts of mRNA corresponding to a nucleic acid molecule that has been inserted into the vector. They can also include a eukaryotic or prokaryotic origin of replication, allowing for autonomous replication within a host cell, sequences that confer resistance to an otherwise toxic drug, thus allowing vector-containing cells to be selected in the presence of the drug, and sequences that increase the efficiency with which the synthesized mRNA is translated. Stable long-term vectors can be maintained as freely replicating entities by using regulatory elements of, for example, viruses (e.g., the OriP sequences from the Epstein Barr Virus genome). Cell lines can also be produced that have the vector integrated into genomic DNA of the cells, and, in this manner, the gene product can be produced in the cells on a continuous basis.

Expression of foreign molecules in bacteria, such as Escherichia coli , requires the insertion of a foreign nucleic acid molecule, e.g., an α1C subunit nucleic acid molecule, into a bacterial expression vector. Such plasmid vectors include several elements required for the propagation of the plasmid in bacteria, and for expression of foreign DNA contained within the plasmid. Propagation of only plasmid-bearing bacteria is achieved by introducing, into the plasmid, a selectable marker-encoding gene that allows plasmid-bearing bacteria to grow in the presence of an otherwise toxic drug. The plasmid also contains a transcriptional promoter capable of directing synthesis of large amounts of mRNA from the foreign DNA. Such promoters can be, but are not necessarily, inducible promoters that initiate transcription upon induction by culture under appropriate conditions (e.g., in the presence of a drug that activates the promoter). The plasmid also, preferably, contains a polylinker to simplify insertion of the gene in the correct orientation within the vector.

Once an appropriate expression vector containing an α1C subunit gene, or a fragment, fusion, or mutant thereof, is constructed, it can be introduced into an appropriate host cell using a transformation technique, such as, for example, calcium phosphate transfection, DEAE-dextran transfection, electroporation, microinjection, protoplast fusion, or liposome-mediated transfection. Host cells that can be transfected with the vectors of this invention can include, but are not limited to, E. coli or other bacteria, yeast, fungi, insect cells (using, for example, baculoviral vectors for expression), or cells derived from mice, humans, or other animals. Mammalian cells can also be used to express α1C subunit proteins using a virus expression system (e.g., a vaccinia virus expression system) described, for example, in Ausubel et al., supra.

In vitro expression of α1C subunit proteins, fusions, polypeptide fragments, or mutants encoded by cloned DNA can also be carried out using the T7 late-promoter expression system. This system depends on the regulated expression of T7 RNA polymerase, an enzyme encoded in the DNA of bacteriophage T7. The T7 RNA polymerase initiates transcription at a specific 23-bp promoter sequence called the T7 late promoter. Copies of the T7 late promoter are located at several sites on the T7 genome, but none are present in E. coli chromosomal DNA. As a result, in T7-infected E. coli , T7 RNA polymerase catalyzes transcription of viral genes, but not E. coli genes. In this expression system, recombinant E. coli cells are first engineered to carry the gene encoding T7 RNA polymerase next to the lac promoter. In the presence of IPTG, these cells transcribe the T7 polymerase gene at a high rate and synthesize abundant amounts of T7 RNA polymerase. These cells are then transformed with plasmid vectors that carry a copy of the T7 late promoter protein.

When IPTG is added to the culture medium containing these transformed E. coli cells, large amounts of T7 RNA polymerase are produced. The polymerase then binds to the T7 late promoter on the plasmid expression vectors, catalyzing transcription of the inserted cDNA at a high rate. Since each E. coli cell contains many copies of the expression vector, large amounts of mRNA corresponding to the cloned cDNA can be produced in this system and the resulting protein can be radioactively labeled.

Plasmid vectors containing late promoters and the corresponding RNA polymerases from related bacteriophages, such as T3, T5, and SP6, can also be used for in vitro production of proteins from cloned DNA. E. coli can also be used for expression using an M13 phage, such as mGPI-2. Furthermore, vectors that contain phage lambda regulatory sequences, or vectors that direct the expression of fusion proteins, for example, a maltose-binding protein fusion protein or a glutathione-S-transferase fusion protein, also can be used for expression in E. coli.

Eukaryotic expression systems are useful for obtaining appropriate post-translational modification of expressed proteins. Transient transfection of a eukaryotic expression plasmid containing an α1C subunit gene, into a eukaryotic host cell allows the transient production of an α1C subunit by the transfected host cell. α1C subunit proteins can also be produced by a stably-transfected eukaryotic (e.g., mammalian) cell line. A number of vectors suitable for stable transfection of mammalian cells are available to the public (see, e.g., Pouwels et al., supra), as are methods for constructing lines including such cells (see, e.g., Ausubel et al., supra).

In one example, cDNA encoding an α1C subunit protein, fusion, mutant, or polypeptide fragment is cloned into an expression vector that includes the dihydrofolate reductase (DIFR) gene. Integration of the plasmid and, therefore, integration of the α1C subunit-encoding gene, into the host cell chromosome is selected for by inclusion of 0.01-300 μM methotrexate in the cell culture medium (Ausubel et al., supra). This dominant selection can be accomplished in most cell types. Recombinant protein expression can be increased by DHFR-mediated amplification of the transfected gene. Methods for selecting cell lines bearing gene amplifications are described in Ausubel et al., supra. These methods generally involve extended culture in medium containing gradually increasing levels of methotrexate. The most commonly used DHFR-containing expression vectors are pCVSEII-DHFR and pAdD26SV(A) (described, for example, in Ausubel et al., supra). The host cells described above or, preferably, a DHFR-deficient CHO cell line (e.g., CHO DHFR-cells, ATCC Accession No. CRL 9096) are among those that are most preferred for DHFR selection of a stably-transfected cell line or DHFR-mediated gene amplification.

Another preferred eukaryotic expression system is the baculovirus system using, for example, the vector pBacPAK9, which is available from Clontech (Palo Alto, Calif.). If desired, this system can be used in conjunction with other protein expression techniques, for example, the myc tag approach described by Evan et al. (Molecular and Cellular Biology 5:3610-3616, 1985).

Once a recombinant protein is expressed, it can be isolated from the expressing cells by cell lysis followed by protein purification techniques, such as affinity chromatography. In this example, an anti-α1C subunit antibody, which can be produced by the methods described herein, can be attached to a column and used to isolate the recombinant α1C subunit proteins. Lysis and fractionation of α1C subunit protein-harboring cells prior to affinity chromatography can be performed by standard methods (see, e.g., Ausubel et al., supra). Once isolated, the recombinant protein can, if desired, be purified further by, e.g., high performance liquid chromatography (HPLC; e.g., see Fisher, Laboratory Techniques In Biochemistry and Molecular Biology, Work and Burdon, Eds., Elsevier, 1980).

Polypeptides of the invention, particularly short α1C subunit fragments and longer fragments of the N-terminus and C-terminus of the a α1C subunit protein, can also be produced by chemical synthesis (e.g., by the methods described in Solid Phase Peptide Synthesis, 2^{nd} ed., 1984, The Pierce Chemical Co., Rockford, Ill.). These general techniques of polypeptide expression and purification can also be used to produce and isolate useful α1C subunit polypeptide fragments or analogs, as described herein.

α1C Subunit Fragments

Polypeptide fragments that include various portions of α1C subunit proteins are useful in identifying the domains of the α1C subunit that are important for its biological activities, such as protein-protein interactions and transcription. Methods for generating such fragments are well known in the art (see, for example, Ausubel et al., supra), using the nucleotide sequences provided herein. For example, an α1C subunit protein fragment can be generated by PCR amplifying a desired α1C subunit nucleic acid molecule fragment using oligonucleotide primers designed based upon α1C subunit nucleic acid sequences. Preferably, the oligonucleotide primers include unique restriction enzyme sites that facilitate insertion of the amplified fragment into the cloning site of an expression vector (e.g., a mammalian expression vector, see above). This vector can then be introduced into a cell (e.g., a mammalian cell; see above) by artifice, using any of the various techniques that are known in the art, such as those described herein, resulting in the production of an α1C subunit polypeptide fragment in the cell containing the expression vector. α1C subunit polypeptide fragments (e.g., chimeric fusion proteins) can also be used to raise antibodies specific for various regions of the α1C subunit using, for example, the methods described below.

α1C Subunit Antibodies

To prepare polyclonal antibodies, α1C subunit proteins, fragments of α1C subunit proteins, or fusion proteins containing defined portions of α1C subunit proteins can be synthesized in, e.g., bacteria by expression of corresponding DNA sequences contained in a suitable cloning vehicle. Fusion proteins are commonly used as a source of antigen for producing antibodies. Two widely used expression systems for E. coli are lacZ fusions using the pUR series of vectors and trpE fusions using the pATH vectors. The proteins can be purified, coupled to a carrier protein, mixed with Freund's adjuvant to enhance stimulation of the antigenic response in an inoculated animal, and injected into rabbits or other laboratory animals. Alternatively, protein can be isolated from α1C subunit-expressing cultured cells. Following booster injections at bi-weekly intervals, the rabbits or other laboratory animals are then bled and the sera isolated. The sera can be used directly or can be purified prior to use by various methods, including affinity chromatography employing reagents such as Protein A-Sepharose, antigen-Sepharose, and anti-mouse-Ig-Sepharose. The sera can then be used to probe protein extracts from α1C subunit-expressing tissue fractionated by polyacrylamide gel electrophoresis to identify α1C subunit proteins. Alternatively, synthetic peptides can be made that correspond to antigenic portions of the protein and used to inoculate the animals.

To generate peptide or full-length protein for use in making, for example, α1C subunit-specific antibodies, an α1C subunit coding sequence can be expressed as a C-terminal or N-terminal fusion with glutathione S-transferase (GST; Smith et al., Gene 67:31-40, 1988). The fusion protein can be purified on glutathione-Sepharose beads, eluted with glutathione, cleaved with a protease, such as thrombin or Factor-Xa (at the engineered cleavage site), and purified to the degree required to successfully immunize rabbits. Primary immunizations can be carried out with Freund's complete adjuvant and subsequent immunizations performed with Freund's incomplete adjuvant. Antibody titers can be monitored by Western blot and immunoprecipitation analyses using the protease-cleaved α1C subunit fragment of the GST-α1C subunit fusion protein. Immune sera can be affinity purified using CNBr-Sepharose-coupled α1C subunit protein. Antiserum specificity can be determined using a panel of unrelated GST fusion proteins.

Alternatively, monoclonal α1C subunit antibodies can be produced by using, as an antigen, α1C subunit protein isolated from α1C subunit-expressing cultured cells or α1C subunit protein isolated from tissues. The cell extracts, or recombinant protein extracts containing α1C subunit protein, can, for example, be injected with Freund's adjuvant into mice. Several days after being injected, the mouse spleens can be removed, the tissues disaggregated, and the spleen cells suspended in phosphate buffered saline (PBS). The spleen cells serve as a source of lymphocytes, some of which would be producing antibody of the appropriate specificity. These can then be fused with permanently growing myeloma partner cells, and the products of the fusion plated into a number of tissue culture wells in the presence of selective agents, such as hypoxanthine, aminopterine, and thymidine (HAT). The wells can then be screened by ELISA to identify those containing cells making antibody capable of binding to an α1C subunit protein, polypeptide fragment, or mutant thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody can be established. The monoclonal antibody can then be purified by affinity chromatography using Protein A Sepharose and ion exchange chromatography, as well as variations and combinations of these techniques. Once produced, monoclonal antibodies are also tested for specific α1C subunit protein recognition by Western blot or immunoprecipitation analysis (see, e.g., Kohler et at., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to GST fusion proteins, peptides corresponding to relatively unique hydrophilic regions of the α1C subunit can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using the α1C subunit, for example, expressed as a GST fusion protein.

Antibodies of the invention can be produced using a α1C subunit amino acid sequences that do not reside within highly conserved regions, and that appear likely to be antigenic, as analyzed by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into the pGEX expression vector. GST fusion proteins can be expressed in E. coli and purified using a glutathione-agarose affinity matrix (Ausubel et al., supra). To generate rabbit polyclonal antibodies, and to minimize the potential for obtaining antisera that is non-specific, or exhibits low-affinity binding to an a α1C subunit, two or three fusions are generated for each protein, and each fusion is injected into at least two rabbits. Antisera are raised by injections in series, preferably including at least three booster injections.

In addition to intact monoclonal and polyclonal anti-α1C subunit antibodies, the invention features various genetically engineered antibodies, humanized antibodies, and antibody fragments, including F(ab′)2, Fab′, Fab, Fv, and sFv fragments. Truncated versions of monoclonal antibodies, for example, can be produced by recombinant methods in which plasmids are generated that express the desired monoclonal antibody fragment(s) in a suitable host. Antibodies can be humanized by methods known in the art, e.g., monoclonal antibodies with a desired binding specificity can be commercially humanized (Scotgene, Scotland; Oxford Molecular, Palo Alto, Calif.). Fully human antibodies, such as those expressed in transgenic animals, are also included in the invention (Green et al., Nature Genetics 7:13-21, 1994).

Ladner (U.S. Pat. Nos. 4,946,778 and 4,704,692) describes methods for preparing single polypeptide chain antibodies. Ward et al., Nature 341:544-546, 1989, describes the preparation of heavy chain variable domains, which they term “single domain antibodies,” and which have high antigen-binding affinities. McCafferty et al., Nature 348:552-554, 1990, show that complete antibody V domains can be displayed on the surface of fd bacteriophage, that the phage bind specifically to antigen, and that rare phage (one in a million) can be isolated after affinity chromatography. Boss et al., U.S. Pat. No. 4,816,397, describes various methods for producing immunoglobulins, and immunologically functional fragments thereof, that include at least the variable domains of the heavy and light chains in a single host cell. Cabilly et al., U.S. Pat. No. 4,816,567, describes methods for preparing chimeric antibodies.

Use of α1C subunit Antibodies

Antibodies to α1C subunit proteins can be used, as noted above, to detect α1C subunit proteins or to inhibit the biological activities of α1C subunit proteins. For example, a nucleic acid molecule encoding an antibody or portion of an antibody can be expressed within a cell to inhibit α1C subunit function. In addition, the antibodies can be coupled to compounds, such as radionuclides and liposomes, for diagnostic or therapeutic uses. Antibodies that specifically recognize extracellular domains of the α1C subunit are useful for targeting such attached moieties to cells displaying such α1C subunit polypeptide domains at their surfaces. Antibodies that inhibit the activity of an a α1C subunit polypeptide described herein can also be useful in preventing or slowing the development of a disease caused by inappropriate expression of a wild type or mutant α1C subunit gene.

Detection of α1C Subunit Gene Expression

As noted, the antibodies described above can be used to monitor α1C subunit protein expression. In situ hybridization of RNA can be used to detect the expression of α1C subunit genes. RNA in situ hybridization techniques rely upon the hybridization of a specifically labeled nucleic acid probe to the cellular RNA in individual cells or tissues. Therefore, RNA in situ hybridization is a powerful approach for studying tissue- and temporal-specific gene expression. In this method, oligonucleotides, cloned DNA fragments, or antisense RNA transcripts of cloned DNA fragments corresponding to unique portions of α1C subunit genes are used to detect specific mRNA species, e.g., in the tissues of animals, such as mice, at various developmental stages. Other gene expression detection techniques are known to those of skill in the art and can be employed for detection of α1C subunit gene expression.

Identification of Additional α1C subunit Genes

Standard techniques, such as the polymerase chain reaction (PCR) and DNA hybridization, can be used to clone α1C subunit homologues in other species and α1C subunit-related genes in humans. α1C subunit-related genes and homologues can be readily identified using low-stringency DNA hybridization or low-stringency PCR with human α1C subunit probes or primers. Degenerate primers encoding human α1C subunit or human α1C subunit-related amino acid sequences can be used to clone additional α1C subunit-related genes and homologues by RT-PCR.

Construction of Transgenic Animals and Knockout Animals

Characterization of α1C subunit genes provides information that allows α1C subunit knockout animal models to be developed by homologous recombination. Preferably, an α1C subunit knockout animal is a mammal, most preferably a mouse. Similarly, animal models of α1C subunit overproduction can be generated by integrating one or more α1C subunit sequences into the genome of an animal, according to standard transgenic techniques. Moreover, the effect of α1C subunit gene mutations (e.g., dominant gene mutations) can be studied using transgenic mice carrying mutated α1C subunit transgenes or by introducing such mutations into the endogenous α1C subunit gene, using standard homologous recombination techniques.

A replacement-type targeting vector, which can be used to create a knockout model, can be constructed using an isogenic genomic clone, for example, from a mouse strain such as 129/Sv (Stratagene Inc., LaJolla, Calif.). The targeting vector can be introduced into a suitably-derived line of embryonic stem (ES) cells by electroporation to generate ES cell lines that carry a profoundly truncated form of an α1C subunit gene. To generate chimeric founder mice, the targeted cell lines are injected into a mouse blastula-stage embryo. Heterozygous offspring can be interbred to homozygosity. α1C subunit knockout mice provide a tool for studying the role of an α1C subunit in embryonic development and in disease. Moreover, such mice provide the means, in vivo, for testing therapeutic compounds for amelioration of diseases or conditions involving an α1C subunit-dependent or an α1C subunit-affected pathway.

Experimental Results

The island beat (isl) mutation perturbs the onset of normal cardiac rhythm in the zebrafish embryo (Stainier et al., Development 123:285-292, 1996; Chen et al., Development 123:293-302, 1996). Despite severe heart defects, island beat zebrafish embryos survive for several days without marked secondary effects, because they receive sufficient oxygen from their environment by diffusion. The island beat alleles, isl^{m458} and isl^{m379} are fully penetrant, and is isl^{m458/m379} trans-heterozygous zebrafish embryos display the same phenotype as homozygotes for any individual mutant allele. In wild-type zebrafish embryos, the primitive heart tube begins to beat by 22 hours post-fertilization (hpf), and to generate blood flow by 24 hpf, with the atrium and the ventricle contracting sequentially (Stainier et al., Trends Cardiovasc. Med. 4:207-212, 1994). The hearts of homozygous mutant isl embryos generate no blood flow, because of functional disturbances in both chambers. In particular, in isl mutant embryos, cells of the ventricle do not beat. Atrial myocytes contract, but in an uncoordinated manner and without evident impulse propagation between neighboring regions of the chamber. The functional appearance of scattered high rate atrial contractions, chamber enlargement, and atrial clot accumulation in isl mutants are characteristics that are reminiscent of the arrhythmia, atrial fibrillation. As in wild-type 48 hpf embryos, electron microscopy reveals developing myofibrillar arrays and cell junctions (i.e., desmosomes and gap junctions) in both atrial and ventricular myocytes of isl embryos.

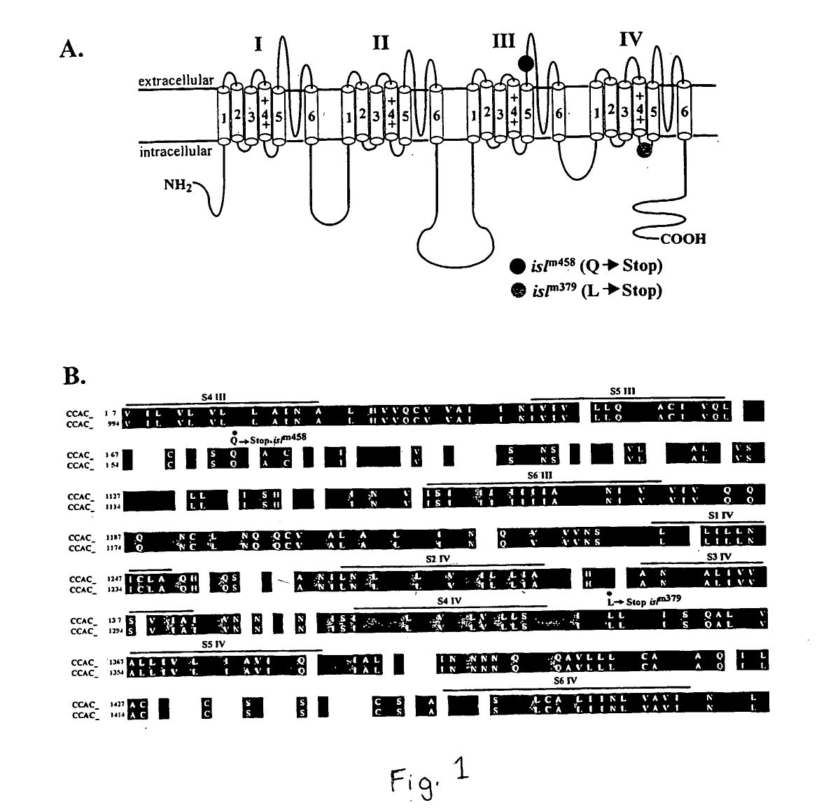

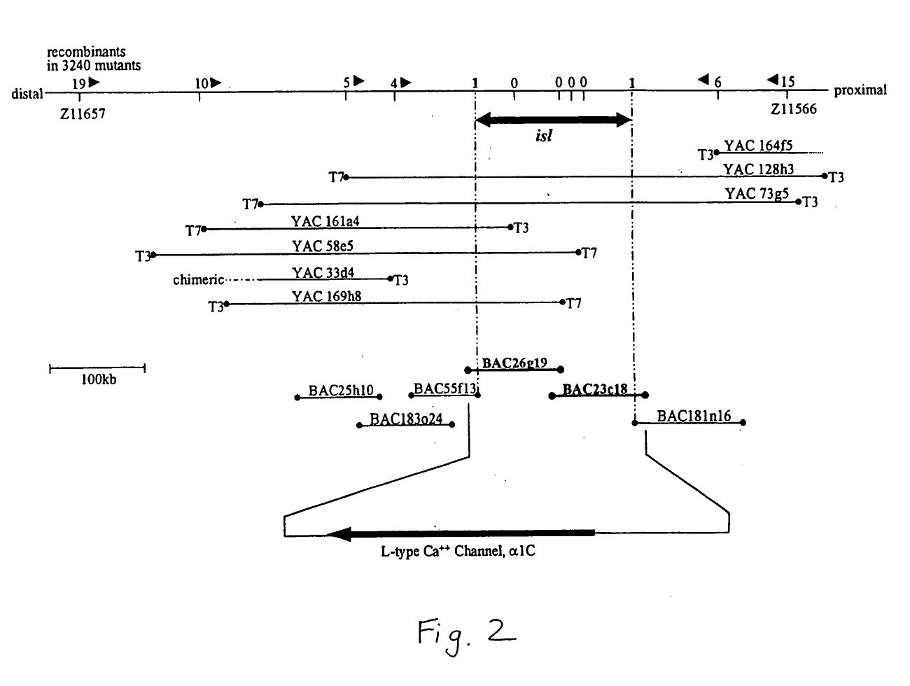

Using microsatellite markers, we mapped isl^{m458} to zebrafish linkage group 4, in a ˜0.6 cM interval between the markers Z11657 and Z11566, which we covered on yeast artificial chromosomes (YACs) and bacterial artificial chromosomes (BACs). We further refined the interval containing the isl gene to BAC clones 26g19 and 23c18, which we sequenced and assembled into contigs (see Methods, below). Only one gene, which is highly homologous to the human voltage-dependent L-type calcium channel α1C subunit (Soldatov, Proc. Natl. Acad. Sci. U.S.A. 89:4628-4632, 1992; Schultz et al., Proc. Natl. Acad. Sci. U.S.A. 90:6228-6232, 1992; FIG. 1A) was identified in these BACs. Conceptual translation of isl genomic DNA and cDNA sequences confirmed high homology (identities =˜78%, positives =84%) to the human voltage-dependent L-type calcium channel α1C subunit (CCAC_human) (FIG. 1B). The zebrafish and human genes encoding the voltage-dependent L-type calcium channel α1C subunit extend over more than 150,000 base pairs (bp) of genomic DNA. Details of the genomic mapping using to identify the zebrafish L-type calcium channel α1 subunit gene are shown in FIG. 2.

To identify the mutations in isl, we sequenced the entire zebrafish voltage-dependent L-type calcium channel α1C subunit (CCAC_zebrafish) coding sequence of the wild-type gene (SEQ ID NOs: 1 and 2) and the two different isl alleles. The isl^{m458} allele has a C-to-T nucleotide transversion at the first base of codon 1077 (CAG->TAG), predicting a change from the amino acid glutamine to a stop codon (Q1077X). Premature termination of translation is predicted to produce a truncated CCAC_zebrafish prior to the transmembrane segment S6 of domain III. In the isl^{m379} allele, a T-to-A nucleotide transversion in codon 1352 (TTG->TAG) changes the amino acid leucine to a stop codon (L1352X), predicting premature termination of translation prior to the pore-forming transmembrane segment S5 of domain IV (FIGS. 1A and 1B).

At the 24-somite stage (21 hpf), when the bilateral cardiac precursors merge to form the primitive heart tube, the α1C subunit of the zebrafish L-type calcium channel is expressed exclusively in the heart. By 26 hpf, isl expression is restricted to the heart tube and the pancreas. isl expression levels and locations are not different between wild-type and isl mutant embryos. From 36 hpf, isl is expressed in certain brain regions (see Methods, below). The expression pattern resembles that described in other species (Iwashima et al., Diabetes 42:948-955, 1993; Takimoto et al., J. Mol. Cell. Cardiol. 29:3035-3042, 1997).

To assess the effect of isl mutations on L-type calcium current, we recorded whole-cell Ca⁺⁺ currents in isl cardiomyocytes (see Methods). Wild-type embryonic zebrafish cardiomyocytes have both voltage-gated L-type and T-type calcium currents (Baker et al., Proc. Natl. Acad. Sci. U.S.A. 94:4554-4559, 1997). In isl^{m458} cardiomyocytes, however, L-type Ca⁺⁺ currents are greatly reduced or absent, while T-type Ca⁺⁺ currents are present.

Hence, the isl phenotype is caused by loss or dramatic reduction in the L-type calcium current in cardiac myocytes, due to mutations in the α1C subunit gene. Effects of mutations in the L-type calcium channel α1C subunit on vertebrate heart function have not been reported. isl mutant zebrafish embryos demonstrate that the L-type calcium current is essential for the embryonic heart beat.

In cardiac cells, calcium influx through the L-type Ca⁺⁺ channel plays an important role in determining action potential characteristics and is responsible for the coupling between excitation and contraction. L-type calcium channels also regulate intracellular Ca⁺⁺ load, and, in this way, determine activity of a number of mitochondrial and cytoplasmic Ca⁺⁺-sensitive enzymes (Carmeliet, Physiol. Rev. 79:917-1017, 1999). T-type calcium currents are believed important in nodal pacemaking (Hermsmeyer et al., Clin. Ther. 19:18-26, 1997), as is the α1D subunit L-type calcium current (Platzer et al., Cell 102:89-97, 2000), and can trigger Ca⁺⁺ release from the sarcoplasmic reticulum, but less efficiently than do L-type Ca⁺⁺ currents (Sipido et al., J. Physiol. (Lond) 508:439-451, 1998; Zhou et al., Biophys. J. 74:1830-1839, 1998).

The chambers of the heart have distinct developmental programs and physiology (Nguyen-Tran et al., Heart Development, Harvey and Rosenthal, Eds., pp. 255-272, 1999), and respond differently to mutation in the α1C subunit. Ventricular cells in isl mutant embryos are silent, and are therefore likely to depend upon calcium current through the L-type calcium channel for initiating contraction. This is similar to autosomal recessive mutations in the L-type calcium channel α1S subunit, which impair skeletal muscle contraction in “muscular dysgenesis” mice (Chaudhari, J. Biol. Chem. 267:25636-25639, 1992). Atrial cells, on the other hand, beat in the isl mutant embryos.

Methods

Mapping and Positional Cloning of isl

Mapping and positional cloning of isl was performed with offspring of the isl^{m458} allele. A genome-wide study of the segregation of microsatellite markers (Knapik et al., Nat. Genet. 18:338-343, 1998) by bulked segregant analysis (Michelmore et al., Proc. Natl. Acad. Sci. U.S.A. 88:9828-9832, 1991) localized isl to the marker Z9247 on linkage group 4. Genetic finemapping placed the isl locus in a ˜0.6 cM interval between the markers Z11657 and Z11566. Nineteen recombinants between isl^{m458} and marker Z11657 and 15 recombinants between and isl^{m458} marker Z11566 were identified after screening 3240 mutant isl^{m458} embryos. YAC clones (Zhong et al., Genomics 48:136-138, 1998) around Z11566 were isolated and ends were oriented by meiotic mapping of mutant isl^{m458} embryos flanking the interval. T7 ends from YACs 128h3 and 73g5 were used to isolate four additional YAC clones 161a4, 58e5, 33d4, and 169h8. A BAC contig was assembled from 6 BACs (Research Genetics) using the different YAC ends as starting points. The length of all BAC clones was determined by pulsed-field gel electrophoresis. Subsequent testing of the BAC ends for recombination events narrowed the interval containing the isl gene to the BAC clones 26g19 and 23c18. These two BACs were shotgun-sequenced, essentially as described (Zhong et al., Science 287:1820-1824, 2000), providing 2.5 fold DNA sequence coverage for each BAC. Sequences were assembled into 18 contigs with the Phred/Phrap/Consed program (http://www.phrap.org/). The voltage-dependent L-type calcium channel α1C subunit was the only gene identified by exon prediction algorithms (GENSCAN, Gene Finder at http://web.wi.mit.edu/bio/pub/) or BLAST search (BLAST 2.0 at http://www.ncbi.nlm.nih.gov/blast/). All exons except exon 1 and exons 46-49 of the zebrafish L-type calcium channel α1C subunit are represented on the genomic DNA contigs.

Sequence Analysis of isl Mutations

The cDNA sequence for CCAC_zebrafish was determined by reverse transcriptase-polymerase reaction (RT-PCR) and rapid amplification of cDNA ends (Clontech). RNA from mutant and wild-type whole embryos or dissected embryonic hearts was isolated using TRIZOL Reagent (Life Technologies). Oligonucleotide sequences were based on sequences of the genomic contigs, and PCR products were cloned with the TOPO TA cloning kit (Invitrogen). Four independent clones were sequenced for each cloned fragment. For the isl^{m458} and isl^{m379} alleles, genomic DNA from mutant, heterozygote, and homozygote embryos was amplified around the point mutations, cloned, and sequenced to confirm the mutations detected. Sequences were aligned, as shown in FIG. 1B, with Pileup Version 10 of the GCG package and displayed by interface of MacBoxshade 2.15 in.

In Situ Analysis of isl Expression

Embryos were staged according to Kimmel et al., Dev. Dyn. 203:253-310, 1995. Whole-mount RNA in situ hybridization was carried out as described (Jowett et al., Trends. Genet. 10:73-74, 1994). A 1224 basepairfragment of cDNA that contains exons 17 through 28 of the zebrafish α1C subunit of the L-type calcium channel was subcloned in pCR II (Invitrogen) for in vitro transcription (Boehringer). RNA probes were digoxygenin-labeled (Boehringer). A Wild M10 dissecting microscope (Leica) equipped with a Nikon camera was used for photomicroscopy.

Determination of the Effect of isl on Calcium Current

Hearts from tricaine-anesthetized, 3 day old embryos were removed, and cells were prepared as described (Baker et al., Proc. Natl. Acad. Sci. U.S.A. 94:4554-4559, 1997). Cardiomyocytes from wild-type line AB were used as controls. Standard whole-cell recordings were performed at room temperature (21-23° C.), essentially as described (Baker et al., Proc. Natl. Acad. Sci. U.S.A. 94:4554-4559, 1997). All data were analyzed blind to genotype. Currents were recorded in Ca⁺⁺ free solutions, with Ba⁺⁺ as a charge carrier to prevent calcium-induced inactivation of the L-type Ca⁺⁺ channel. L-type calcium channels passing Ba⁺⁺ current are evident as an inward current that appears when the membrane voltage is stepped from a holding voltage of −60 mV to voltages more positive than −30 mV. With Ba⁺⁺ as the charge carrier, the current decreases slowly over time. The bath solution contained 15 mM BaCl₂, 5 mM CsCl, 10 mM Hepes, 10 mM glucose, and 125 mM N-methyl-D-glucamine (pH 7.4, with HCl). The pipette solution contained 140 mM tetraethylammonium-chloride, 0.5 mM MgCl₂, 10 mM Hepes, 10 mM EGTA, 5 mM mGatp, and 0.1 mM Camp (pH 7.4, with CsOH). Pipettes were fire-polished to resistances of 2-5 MO. Data acquisition and analysis were carried out using pClamp6 software (Axon Instruments). P Current traces were obtained from a single wild-type (WT) and isl^{m458} cardiomyocyte and were recorded with barium (Ba⁺⁺) as a charge carrier. Representative currents were obtained by holding the cell membrane at either −60 mV (V_{H}−60 mV) or −100 mV (V_{H}−100 mV) and depolarizing the membrane in 10 mV steps, beginning at −50 mV and ending at +10 mV. Current traces were filtered at 1 kHz. Linear leak currents were determined by a small depolarizing step, and subtracted off all subsequent records. To maximize the separation of L- and T-type calcium currents, we took advantage of their different responses to voltage. T-type currents are largest when the cell membrane is held at −100 mV and the test voltage is −30 mV. L-type current is isolated when the cell is held at a depolarized voltage of −60 mV to inactivate the T-type channels. The maximum L-type currents are then elicited by stepping the membrane to −10 mV. Holding the cells at 60 mV and depolarizing to −10 mV showed that the average inward current obtained after 400 ms was significantly larger (−18 pA±5.2 pA SEM) in wild-type cells (n=13), than in isl^{m458} cells (n=13) (−5.6 pA±3.0 pA SEM) (p<0.05). Similar results were obtained with a holding voltage of −100 mV.

Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it is to be understood that it is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the appended claims.

What is claimed is:

1. A method of determining whether a test subject has, or is at risk of developing, a disease or condition related to an α1C subunit of a voltage-dependent L-type calcium channel, said method comprising analyzing a nucleic acid molecule of a sample from the test subject to determine whether the test subject has a mutation in a gene encoding said subunit, wherein the presence of a mutation indicates that said test subject has, or is at risk of developing, a disease related to an α1C subunit of a voltage-dependent L-type calcium channel.

2. The method of claim 1, further comprising the step of using nucleic acid molecule primers specific for a gene encoding the α1C subunit of a voltage-dependent L-type calcium channel for nucleic acid molecule amplification of the gene by the polymerase chain reaction.

3. The method of claim 1, wherein determination of whether said gene comprises a mutation is carried out by sequencing a nucleic acid molecule encoding an α1C subunit of a voltage-dependent L-type calcium channel from said test subject.

4. The method of claim 1, wherein said test subject is a mammal.

5. The method of claim 1, wherein said test subject is human.

6. The method of claim 1, wherein said disease or condition is heart disease.

7. The method of claim 6, wherein said heart disease is cardiac arrhythmia.

8. The method of claim 7, wherein said cardiac arrhythmia is atrial fibrillation.

9. The method of claim 1, wherein said mutation is the island beat mutation.

10. A method for identifying a compound that can be used to treat or to prevent heart disease, said method comprising contacting an organism comprising a mutation in a gene encoding an α1C subunit of a voltage-dependent L-type calcium channel and having a phenotype characteristic of heart disease with said compound, and determining the effect of said compound on said phenotype, wherein detection of an improvement in said phenotype indicates the identification of a compound that can be used to treat or to prevent heart disease.

11. The method of claim 10, wherein said heart disease is cardiac arrhythmia.

12. The method of claim 11, wherein said cardiac arrhythmia is atrial fibrillation.

13. The method of claim 10, wherein said organism is a zebrafish.

14. The method of claim 10, wherein said mutation in the gene encoding the α1C subunit of a voltage-dependent L-type calcium channel is the island beat mutation.

15. A method of treating or preventing heart disease in a patient, said method comprising administering to said patient a compound identified using the method of claim 10.

16. The method of claim 15, wherein said heart disease is cardiac arrhythmia.

17. The method of claim 15, wherein said heart disease is atrial fibrillation.

18. The method of claim 15, wherein said patient has a mutation in a gene encoding an α1C subunit of a voltage-dependent L-type calcium channel.

19. The method of claim 15, wherein said mutation is the island beat mutation.

20. A method of treating or preventing heart disease in a patient, said method comprising administering to said patient a functional α1C subunit of a voltage-dependent L-type calcium channel or an expression vector comprising a nucleic acid molecule encoding said subunit.

21. A substantially pure zebrafish α1C subunit of a voltage-dependent L-type calcium channel.

22. The polypeptide of claim 21, wherein said polypeptide comprises an amino acid sequence that is substantially identical to the amino acid sequence of SEQ ID NO:2.

23. The polypeptide of claim 22, wherein said polypeptide comprises the amino acid sequence of SEQ ID NO:2.

24. A substantially pure nucleic acid molecule comprising a sequence encoding a zebrafish α1C subunit of a voltage-dependent L-type calcium channel.

25. The nucleic acid molecule of claim 24, wherein said nucleic acid molecule encodes a polypeptide comprising an amino sequence that is substantially identical to the amino acid sequence of SEQ ID NO:2.

26. The nucleic acid molecule of claim 25, wherein said nucleic acid molecule encodes a polypeptide comprising the amino acid sequence of SEQ ID NO:2.

27. The nucleic acid molecule of claim 24, wherein said nucleic acid molecule is DNA.

28. A vector comprising the nucleic acid molecule of claim 24.

29. A cell comprising the vector of claim 28.

30. A non-human transgenic animal comprising the nucleic acid molecule of claim 24.

31. The non-human transgenic animal of claim 30, wherein said animal is a zebrafish.

32. A non-human animal having a knockout mutation in one or both alleles encoding a α1C subunit polypeptide.

33. A cell from the non-human knockout animal of claim 32.

34. A non-human transgenic animal comprising a nucleic acid molecule encoding a mutant α1C subunit of a voltage-dependent L-type calcium channel.

35. The non-human transgenic animal of claim 34, wherein the non-human transgenic animal is a zebrafish.

36. The non-human transgenic animal of claim 35, wherein the non-human transgenic animal comprises the island beat mutation.

37. An antibody that specifically binds to an α1C subunit of a voltage-dependent L-type calcium channel.

38. Use of a compound identified using the method of claim 10 in the preparation of a medicament for treating or preventing heart disease in a patient.

39. Use of a α1C subunit of a voltage-dependent L-type calcium channel or an expression vector comprising a nucleic acid molecule encoding said subunit in the preparation of a medicament for treating or preventing heart disease in a patient.
